(19) United States
(12) Patent Application Publication
(10) Pub. No.: US 2010/0167413 A1
(43) Pub. Date: Jul. 1, 2010

(54) METHODS AND SYSTEMS FOR ANALYZING FLUORESCENT MATERIALS WITH REDUCED AUTOFLUORESCENCE

(76) Inventors: Paul Lundquist, San Jose, CA (US); Cheng Frank Zhong, Sunnyvale, CA (US); Denis Zaccarin, San Jose, CA (US); Pegian Zhao, Mountain View, CA (US); Stephen Turner, Menlo Park, CA (US)

Correspondence Address: PACIFIC BIOSCIENCES OF CALIFORNIA, INC., 1505 ADAMS DR., MENLO PARK, CA, 94025, US

(21) Appl. No.: 12/451,310

(22) PCT Filed: May 9, 2008
(86) PCT No.: PCT/US08/05953

§ 371 Date: Mar. 3, 2010

Related U.S. Application Data
(63) Continuation-in-part of application No. 11/901,273, filed on Sep. 14, 2007.
(60) Provisional application No. 60/928,617, filed on May 10, 2007.
(60) Provisional application No. 60/928,617, filed on May 10, 2007.

(51) Int. Cl. G01N 21/64; C12M 1/00

(52) U.S. Cl. 436/172; 422/82.08; 435/287.2

(57) ABSTRACT

Mitigative and remedial approaches to reduction of autofluorescence background noise are applied in analytical systems that rely upon sensitive measurement of fluorescent signals from arrays of fluorescent signal sources. Such systems are for particular use in fluorescence based sequencing by incorporation systems that rely upon small numbers or individual fluorescent molecules in detecting incorporation of nucleotides in primer extension reactions. Systems and methods for analyzing highly multiplexed sample arrays using highly multiplexed, high-density optical systems to illuminate high-density sample arrays and/or provide detection and preferably confocal detection off signals emanating from such high-density arrays. Systems and methods are applied in a variety of different analytical operations, including analysis of biological and biochemical reactions, including nucleic acid synthesis and derivation of sequence information from such synthesis.

### CROSS-REFERENCE TO RELATED APPLICATIONS

The present application claims priority from Provisional U.S. Patent Application No. 60/928,617, filed May 10, 2007, and benefit of U.S. patent application Ser. No. 11/901,273, filed Sep. 14, 2007, the full disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

Not Applicable.

### FIELD OF THE INVENTION

The invention is in the field of reducing autofluorescence background noise.

### BACKGROUND OF THE INVENTION

Typical fluorescence based optical analysis of analytical reactions employs reactants or other reagents in the reaction of interest that bear a fluorescent moiety, such as a labeling group, where the detection of that moiety is indicative of a particular reaction result or condition. For example, reactions may be engineered to produce a change in the amount, location, spectrum, or other characteristic upon occurrence of a reaction of interest.

During analysis, an excitation light source is directed through an optical system or train at the reaction to excite fluorescence from the fluorescent moiety. The emitted fluorescence is then collected by the optical train and directed toward a detection system, which quantifies, records, and/or processes the signal data from the fluorescence. Fluorescence-based systems are generally desired for their high signal levels deriving from the high quantum efficiency of the available fluorescent dye moieties. Because of these high signal levels, relatively low levels of the materials are generally required in order to observe a fluorescent signal.

For example, simple multi-well plate readers have been ubiquitously employed in analyzing optical signals from fluid based reactions that were being carried out in the various wells of a multiwell plate. These readers generally monitor the fluorescence, luminescence or chromogenic response of the reaction solution that results from a given reaction in each of 96, 384 or 1536 different wells of the multiwell plate.

Other optical detection systems have been developed and widely used in the analysis of analytes in other configurations, such as in flowing systems, i.e., in the capillary electrophoretic separation of molecular species. Typically, these systems have included a fluorescence detection system that directs an excitation light source, e.g., a laser or laser diode, at the capillary, and is capable of detecting when a fluorescent or fluorescently labeled analyte flows past the detection region (see, e.g., ABI 3700 Sequencing systems, Agilent 2100 Bioanalyzer and ALP systems, etc.). Other detection systems direct a scanning laser at surface bound analytes to determine where, on the surface, the analytes have bound. Such systems are widely used in molecular array based systems, where the positional binding of a given fluorescently labeled molecule on an array indicates a characteristic of that molecule, e.g., complementarity or binding affinity to a given molecule (See, e.g., U.S. Pat. No. 5,578,832).

Notwithstanding the great benefits of fluorescent reaction systems, the development of real-time, highly multiplexed, single molecule analyses and the application of these systems does have some drawbacks particularly when used in extremely low signal level reactions, e.g., low concentration or even single molecule detection systems. In particular, these systems often have a number of components that can potentially generate amounts of background signal, e.g., detected signal that does not emanate from the fluorescent species of interest, when illuminated with relatively high intensity radiation. This background signal can contribute to signal noise levels, and potentially overwhelm relatively low reaction derived signals or make more difficult the identification of signal events, e.g., increases, decreases, pulses etc., of fluorescent signal associated with the reactions being observed.

Background signal, or noise, can derive from a number of sources, including, for example, fluorescent signals from non targeted reaction regions, fluorescence from targeted reaction regions but that derive from non-relevant sources, such as non-specific reactions or associations, such as dye or label molecules that have nonspecifically adsorbed to surfaces, prevalence or build up of labeled reaction products, other fluorescent reaction components, contaminants, and the like. Other sources of background signals in fluorescent systems include signal noise that derives from the use of relatively high-intensity excitation radiation in conjunction with sensitive light detection. Such noise sources include those that derive from errant light entering the detection system that may come from inappropriately filtered or blocked excitation radiation, and/or contaminating ambient light sources that may impact the overall system. Other sources of signal noise resulting from the application of high intensity excitation illumination derives from the auto-fluorescence of the various components of the system when subjected to such illumination, as well as Raman scattering of the excitation illumination. The contribution of this systemic fluorescence is generally referred to herein as autofluorescence background noise (ABN).

It would be therefore desirable to provide methods, components and systems in which background signal, such as autofluorescence background noise, were minimized. This is particularly the case in relatively low signal level reactions, such as single molecule fluorescence detection methods and systems, e.g., real-time, highly multiplexed single molecule detection systems that are capable of detecting large numbers of different events at relatively high speed and that are capable of deconvolving complex, multi-wavelength signals. The present invention meets these and other needs.

### SUMMARY OF THE INVENTION

The invention provides methods and systems that have improved abilities to monitor fluorescent signals from analytical reactions by virtue of having reduced levels of background signal noise that derives from autofluorescence created within one or more components of the overall system.

In a first aspect, the invention provides systems for monitoring a plurality of discrete fluorescent signals from a substrate. The systems include a substrate onto which a plurality of discrete fluorescent signal sources has been disposed, an excitation illumination source, and a detector for detecting fluorescent signals from the plurality of fluorescent signal sources. In addition, the systems include an optical train positioned to simultaneously direct excitation illumination from the excitation illumination source to each of the plurality of discrete fluorescent signal sources on the substrate and direct fluorescent signals from the plurality of fluorescent signal sources to the detector. The optical train of the systems comprises an objective lens focused in a first focal plane at the substrate for simultaneously collecting fluorescent signals from the plurality of fluorescent signal sources on the substrate, a first focusing lens for receiving the fluorescent signals from the objective lens and focusing the fluorescent signals in a second focal plane, and a confocal filter placed within the second focal plane to filter fluorescent signals from the substrate that are not within the first focal plane.

Optionally, the systems for monitoring a plurality of discrete fluorescent signals from a substrate can include a substrate that comprises first and second opposing surfaces that is positioned such that the first surface of the substrate is more proximal to the optical train than the second surface, and such that the first focal plane is substantially coplanar with the second surface. The systems can optionally include an optical train that simultaneously directs excitation radiation at and collects fluorescent signals from at least 100 discrete fluorescent signal sources, at least 500 discrete fluorescent signal sources, at least 1000 discrete signal sources, or at least 5000 discrete signal sources. The systems can optionally include an optical train that comprises a microlens array and/or a diffractive optical element to simultaneously direct excitation illumination at the plurality of discrete fluorescent signal sources on the substrate.

Each of the plurality of discrete signal sources in the systems described above can optionally comprise a reaction region, e.g., an optically confined region on the substrate, into which a complex comprising a nucleic acid polymerase, a template sequence, and a primer sequence, and at least one fluorescently labeled nucleotide has been disposed. Optionally, the optically confined regions can comprise zero mode waveguides.

The invention also provides second set of systems for monitoring a plurality of discrete fluorescent signals from a substrate, which includes a substrate onto which a plurality of discrete fluorescent signal sources has been disposed, an excitation illumination source, and a detector for detecting fluorescent signals from the plurality of fluorescent signal sources. In addition, the second set of systems of monitoring a plurality of discrete fluorescent signals from a substrate includes an optical train that is positioned to direct excitation illumination from the excitation illumination source to each of the plurality of discrete fluorescent signal sources on the substrate in a targeted illumination pattern. In addition, the optical train directs fluorescent signals from the plurality of fluorescent signal sources to the detector.

Optionally, the optical train in the second set systems for monitoring a plurality of discrete fluorescent signals from a substrate can comprise a microlens array and/or a diffractive optical element to direct excitation radiation to each of the plurality of discrete fluorescent signal sources in a targeted illumination pattern. The diffractive optical element can optionally be configured to direct excitation radiation to at least 100 discrete fluorescent signal sources, at least 500 discrete fluorescent signal sources, at least 1000 discrete fluorescent signal sources, or at least 5000 discrete fluorescent signal sources in a targeted illumination pattern.

In the second set systems for monitoring a plurality of discrete fluorescent signals from a substrate, each of the plurality of discrete signal sources can optionally comprise a reaction region, e.g., an optically confined region on the substrate, into which a complex comprising a nucleic acid polymerase, a template sequence, and a primer sequence, and at least one fluorescently labeled nucleotide has been disposed. The optically confined regions can optionally comprise zero mode waveguides.

In a related aspect, the invention provides methods of reducing fluorescence background signals in detecting fluorescent signals from a substrate that comprises a plurality of fluorescent signal sources. The methods include directing excitation radiation simultaneously at a plurality of fluorescent signal sources on a substrate in a first focal plane, collecting fluorescent signals simultaneously from the plurality of fluorescent signal sources, filtering the fluorescent signals to reduce fluorescence not in the first focal plane to provide filtered fluorescent signals, and detecting the filtered fluorescent signals. The filtering step in the methods can optionally comprise confocally filtering the fluorescent signals to provide filtered fluorescent signals.

The invention also provides methods of detecting fluorescent signals from a plurality of discrete fluorescent signal sources on a substrate. These methods include providing a substrate onto which a plurality of discrete fluorescent signal sources has disposed, directing excitation illumination at the substrate in a targeted illumination pattern, and detecting fluorescent signals from each of the plurality of discrete fluorescent signal sources. The step of directing excitation at the substrate in a targeted illumination pattern can optionally comprise passing the excitation illumination through a microlens array and/or a diffractive optical element. The targeted illumination pattern can optionally comprise at least 100 discrete illumination spots positioned to be incident upon at least 100 discrete fluorescent signal sources, at least 500 discrete illumination spots positioned to be incident upon at least 500 discrete fluorescent signal sources, at least 1000 discrete illumination spots positioned to be incident upon at least 1000 discrete fluorescent signal sources, or at least 5000 discrete illumination spots positioned to be incident upon at least 5000 discrete fluorescent signal sources.

In addition, the invention provides three sets of methods of monitoring fluorescent signals from a source of fluorescent signals. In the first set, the methods include providing a fluorescent signal detection system that comprises a substrate comprising a plurality of discrete fluorescent signal sources, providing a source of excitation illumination, providing a fluorescent signal detector, and providing an optical train for directing excitation illumination from the source of excitation illumination to the substrate and for directing fluorescent signals from the substrate to the fluorescent signal detector. In this set of methods, at least one optical component in the optical train is photobleached so as to reduce a level of autofluorescence produced by the at least one optical component in response to passing excitation illumination therethrough.

The second set of methods of monitoring fluorescent signals from a source of fluorescent signals includes providing a substrate onto which a plurality of discrete fluorescent signal sources have been disposed, directing excitation illumination at the substrate in a targeted illumination pattern to excite fluorescent signals from the fluorescent signal sources, collecting the fluorescent signals from the plurality of discrete fluorescent signal sources illuminated with the targeted illumination pattern, confocally filtering the fluorescent emissions, and separately detecting the fluorescent emissions from the discrete fluorescent signal sources.

The third set of methods of monitoring fluorescent signals from a source of fluorescent signals includes providing an excitation illumination source, providing a substrate onto which at least a first fluorescent signal source has been disposed, and providing an optical train comprising optical components that is positioned to direct excitation illumination from the illumination source to the at least first fluorescent signal source and for transmitting fluorescent signals from the at least first fluorescent signal source to a detector. The third set of methods includes photobleaching at least one of the optical components to reduce an amount of autofluorescence produced by the at least one optical component in response to the excitation illumination, directing excitation illumination through the at least one optical component and at the at least first fluorescent signal source, and detecting fluorescent signals from the at least first fluorescent signal source. In the third set of methods, the fluorescent signals can optionally be confocally filtered prior to being detected.

Relatedly, the invention provides systems for detecting fluorescent signals from a plurality of signal sources on a substrate. These systems include a source of excitation illumination, a detection system, and an optical train positioned to direct excitation illumination from the source of excitation illumination to the plurality of signal sources on the substrate and transmit emitted fluorescence from the plurality of fluorescent signal sources to the detector. The optical train in these systems includes an objective lens that has a ratio of excitation illumination to autofluorescence of greater than 1×10⁻¹⁰.

The present invention is generally directed to highly multiplexed optical interrogation systems, and particularly to highly multiplexed fluorescence-based detection systems. In one aspect, the present invention is directed at systems and methods for high resolution, highly multiplexed analysis of optical signals from large numbers of discrete signal sources, and particularly signal sources that are of very small dimensions and which are arrayed on or within substrates at regularly spaced intervals.

In a first aspect, the invention includes multiplex fluorescence detection systems that comprise an excitation illumination source, and an optical train that comprises an illumination path and a fluorescence path. In the context of certain aspects of the invention, the illumination path comprises an optical train that comprises multiplex optics that convert a single originating illumination beam from the excitation illumination source into at least 10 discrete illumination beams, and an objective lens that focuses the at least 10 discrete illumination beams onto at least 10 discrete locations on a substrate. The fluorescence path comprises collection and transmission optics that receive fluorescent signals from the at least 10 discrete locations, and separately direct the fluorescent signals from each of the at least 10 discrete locations through a confocal filter and focus the fluorescent signals onto a different location on a detector.

In a related aspect, the invention provides a system for detecting fluorescence from a plurality of discrete locations on a substrate, which system comprises a substrate, an excitation illumination source a detector, and an optical train positioned to receive an originating illumination beam from the excitation illumination source. In the context of certain aspects of the invention, the optical train is configured to convert the originating illumination beam into a plurality of discrete illumination beams, and focus the plurality of discrete illumination beams onto a plurality of discrete locations on the substrate, wherein the plurality of discrete locations are at a density of greater than 1000 discrete illumination spots per mm², preferably greater than 10,000 discrete spots per mm², more preferably greater than 100,000 discrete illumination spots per mm², in many cases greater than 250,000 discrete illumination spots per mm², and in some cases up to and greater than 1 spot per μm². In terms of inter-spot spacing upon the substrate, the illumination patterns of the invention will typically provide spacing between adjacent spots (in the closest dimension), of less than 100 μm, center to center, preferably, less than 20 μm, more preferably, less than 10 μm, and in many preferred cases, spacing between spots of 1 μm or less, center to center. As will be appreciated, such spacing generally refers to inter-spot spacing in the closes dimension, and does not necessarily reflect inter-row spacing that may be substantially greater, due to the allowed spacing for spectral separation of adjacent rows, as discussed elsewhere herein. The optical train is further configured to receive a plurality of discrete fluorescent signals from the plurality of discrete locations, and focus the plurality of discrete fluorescent signals through a confocal filter, onto the detector.

In other aspects, the invention provides systems for collecting fluorescent signals from a plurality of locations on a substrate, which comprise excitation illumination optics configured to simultaneously provide excitation radiation to an area of a substrate that includes the plurality of locations, and fluorescence collection and transmission optics that receive fluorescent signals from the plurality of locations on the substrate, and separately direct the fluorescent signals from each of the plurality of locations through a separate confocal aperture in a confocal filter and image the fluorescent signals onto a detector.

Relatedly, the invention also provides systems for detecting fluorescent signals from a plurality of discrete locations on a substrate, that comprise an excitation illumination source, a diffractive optical element or holographic phase mask, positioned to convert a single originating illumination beam from the excitation illumination source into at least 10 discrete beams each propagating at a unique angle relative to the originating beam, an objective for focusing the at least ten discrete beams onto at least 10 discrete locations on a substrate, fluorescence collection and transmission optics, and a detector. In the context of certain aspects of the invention, the fluorescence collection and transmission optics are positioned to receive fluorescent signals from the plurality of discrete locations and transmit the fluorescent signals to the detector.

In other aspects, the invention provides methods of detecting a plurality of discrete fluorescent signals from a plurality of discrete locations on a substrate. The methods comprise simultaneously and separately illuminating each of the plurality of discrete locations on the substrate with excitation illumination. Fluorescent signals from each of the plurality of locations are simultaneously and separately collected and each of the fluorescent signals from the plurality of discrete locations is separately directed through a confocal filter, and separately imaged onto a discrete location on a detector.

Those of skill in the art will appreciate that that the methods provided by the invention, e.g., for detecting a plurality of discrete fluorescent signals from a plurality of discrete locations on a substrate, for reducing fluorescence background signals in detecting fluorescent signals from a substrate that comprises a plurality of fluorescent signal sources, and/or for monitoring fluorescent signals from a source of fluorescent signals, can be used alone or in combination and can be used in combination with any one or more of the systems described herein. Likewise, the systems provided by the invention, e.g., multiplex fluorescence detection systems, systems for monitoring a plurality of discrete fluorescent signals from a substrate, and/or systems for detecting fluorescence from a plurality of discrete locations on a substrate, can be used alone or in combination. In addition to the foregoing, the invention is also directed to the use of any of the foregoing systems and/or methods in a variety of analytical operations.

### BRIEF DESCRIPTION OF THE DRAWINGS

FIG. 1 provides a schematic overview of a fluorescence detection system.

FIG. 2 shows a plot of fluorescent signals as a function of the number of illumination lines applied to a given fluorescently spotted substrate, showing increasing background fluorescence levels with increasing illumination.

FIG. 3 schematically illustrates a targeted illumination pattern generated from an originating beam passed through differently oriented diffraction gratings.

FIG. 4 provides an example of a microlens array for use in the present invention.

FIG. 5 shows an image of diffractive optical element (“DOE”) and the illumination pattern generated when light is passed through the DOE.

FIG. 6 shows an illumination pattern from a DOE designed to yield very high illumination multiplex.

FIG. 7 schematically illustrates a targeted illumination pattern generated from overlaying illumination patterns from two DOEs but offsetting them by a half period.

FIG. 8 schematically illustrates an illumination path including a polarizing beam splitting element.

FIG. 9 shows a comparison plot of autofluorescence of a fluorescent detection system in the absence and presence of a confocal mask in the system, to filter out of focus autofluorescence components.

FIG. 10 schematically illustrates a portion of a confocal mask.

FIG. 11 provides a schematic of an optical train incorporating a confocal mask.

FIG. 12 is a comparative plot of autofluorescence imaged at a discrete detector location in the absence of a confocal mask, and in the presence of confocal slits of decreasing cross sectional dimensions.

FIG. 13 provides a schematic illustration of a fluorescence detection system that can be used with the methods and systems of the present invention.

FIG. 14 schematically illustrates the illumination and fluorescence paths of one exemplary system according to the invention.

### DETAILED DESCRIPTION

### I. General Discussion of Invention

The present invention generally provides methods, processes and systems for monitoring fluorescent signals associated with reactions of interest, but in which background signal levels and particularly autofluorescence background noise of system components, is reduced.

The methods, processes and systems of the invention are particularly suited to the detection of fluorescent signals from signal sources, e.g., reaction regions, on substantially planar substrates, and particularly for detection of relatively low levels of fluorescent signals from such reaction regions, where signal background has a greater potential for negative impact.

Increasing throughput of chemical, biochemical and/or biological analyses has generally relied, at least in part, on the ability to multiplex the analysis. Accordingly, in a preferred embodiment, the methods, processes and systems of the invention can be used with multiplexed optical systems for high-throughput analysis of fluorescent signal sources, e.g., fluorescent signal sources associated with chemical, biochemical, or biological reactions. Such multiplex generally utilizes the simultaneous analysis of multiple different samples that are either physically discrete or otherwise separately identifiable within the analyzed material. Examples of such multiplex analysis include, e.g., the use of multi-well plates and corresponding plate readers, to optically interrogate multiple different fluorescent reactions simultaneously. Such plate systems have been configured to include 16 wells, 32 wells, 96 wells, 384 wells and even 1536 wells in a single plate that can be interrogated simultaneously.

Multiplexed systems in which autofluorescence background noise is beneficially minimized, e.g., by the methods and systems of the invention, include array based technologies in which solid substrates bearing discrete patches of different molecules are reacted with a certain set of reagents and analyzed for reactivity, e.g., an ability to generate a fluorescent signal. Such arrays are simultaneously interrogated with the reagents and then analyzed to identify the reactivity of such reagents with the different reagents immobilized upon different regions of the substrate.

In the context of the present invention, the optical signal sources that are analyzed using the methods and systems typically can comprise any of a variety of materials, and particularly those in which optical analysis may provide useful information. Of particular relevance to the present invention are optical signal sources that comprise chemical, biochemical or biological materials that can be optically analyzed to identify one or more chemical, biochemical and/or biological properties. Such materials include chemical or biochemical reaction mixtures that may be analyzed to determine reactivity under varying conditions, varying reagent concentrations, exposure to different reagents, or the like. Examples of materials of particular interest include proteins such as enzymes, their substrates, antibodies and/or antigens, biochemical pathway components, such as receptors and ligands, nucleic acids, including complementary nucleic acid associations, nucleic acid processing systems, e.g., ligases, nucleases, polymerases, and the like. These materials may also include higher order biological materials, such as prokaryotic or eukaryotic cells, mammalian tissue samples, viral materials, or the like.

Optical interrogation or analysis of these materials can generally involve known optical analysis concepts, such as analysis of light absorbance, transmittance and/or reflectance of the materials being analyzed. In other aspects, such analysis may determine a level of optical energy emanating from the system. In some cases, material systems may produce optical energy, or light, as a natural product of the process being monitored, as is the case in systems that use chemiluminescent reporter systems, such as pyrosequencing processes (See, e.g., U.S. Pat. No. 6,210,891). In particularly preferred aspects, the optical analysis of materials in accordance with the present invention comprises analysis of the materials' fluorescent characteristics, e.g., the level of fluorescent emissions emanating from the material in response to illumination with an appropriate excitation radiation. Such fluorescent characteristics may be inherent in the material being analyzed, or they may be engineered or exogenously introduced into the system being analyzed. By way of example, the use of fluorescently labeled reagent analogs in a given system can be useful in providing a fluorescent signal event associated with the reaction or process being monitored.

In certain aspects, the optical signal sources analyzed using methods, processes, and systems provided by the invention are referred to as being provided on a substrate. Such substrates may comprise any of a wide variety of supporting substrates upon which such signal sources may be deposited or otherwise provided, depending upon the nature of the material and the analysis to be performed. For example, in the case of fluid reagents, such substrates may comprise a plate or substrate bearing one or more reaction wells, where each fluorescent signal source may comprise a discrete reaction well on the plate, or even a discrete region within a given reaction well. In terms of multi-well plates, as noted above, such plates may comprise a number of discrete and fluidically isolated reaction wells. In fact, such plates are generally commercially available in a variety of formats ranging from 8 wells, to 96 wells, to 384 wells to 1536 wells, and greater. In certain aspects, each discrete well on a multi-well plate may be considered a discrete, e.g., fluorescent, signal source. However, in some aspects, a single well may include a number of discrete fluorescent signal sources. As used herein, a discrete fluorescent signal source typically denotes a fluorescent signal source that is optically resolvable and separately identifiable from another adjacent fluorescent signal source. Such separate identification may be a result of different chemical or biochemical characteristics of each fluorescent signal source or merely result from spatial differentiation between fluorescent signal sources.

Other substrates that can be used with the methods and systems of the invention, particularly in the field of biochemical analysis, include planar substrates upon which are provided arrays of varied molecules, e.g., proteins or nucleic acids. In such cases, different features on the array, e.g., spots or patches of a given molecule type, may comprise a discrete signal source.

The methods and systems of the invention are generally applicable to a wide variety of multiplexed analysis of a number of discrete optical signal sources on a substrate. Of particular benefit in the present invention is its applicability to extremely high-density arrays of such optical signal sources and/or arrays of such signal sources where each signal source is of extremely small area and/or signal generating capability. Examples of such arrayed signal sources include, for example, high density arrays of molecules, e.g., nucleic acids, high density multi-well reaction plates, arrays of optical confinements, and the like.

For ease of discussion, the present invention is described in terms of its application to multiplexed arrays of single molecule reaction regions on planar substrates from which fluorescent signals emanate, which signals are indicative of a particular reaction occurring within such reaction regions. Though described in terms of such single molecule arrays, it will be appreciated that the invention, as a whole, or in part, will have broader applicability and may be employed in a number of different applications, such as in detection of fluorescent signals from other array formats, e.g., spotted arrays, arrays of fluidic channels, conduits or the like, or detection of fluorescent signals from multi-well plate formats, fluorescent bar-coding techniques, and the like.

One exemplary analytical system or process in which the invention is applied is in a single molecule DNA sequencing operation in which an immobilized complex of DNA polymerase, DNA template and primer are monitored to detect incorporation of nucleotides or nucleotide analogs that bear fluorescent detectable groups. See, e.g., U.S. Pat. Nos. 7,033,764, 7,052,847, 7,056,661, and 7,056,676, the disclosures of which are incorporated herein by reference in their entirety for all purposes. In brief, these arrays typically comprise a transparent substrate e.g., glass, quartz, fused silica, or the like, having an opaque, e.g., typically a metal, layer disposed over its surface. A number of apertures are provided in the metal layer through to the transparent substrate. In waveguide nomenclature, the apertures are typically referred to as cores, while the metal layer functions as the cladding layer. Typically, large numbers of cores are provided immobilized upon the substrates, and positioned such that individual biological or biochemical complexes are optically resolvable when associated with a fluorescent labeling group or molecule, such as a labeled nucleotide or nucleotide analog.

In preferred aspects, e.g., that maximize throughput of the sequencing process, the individual complexes may be provided within an optically confined space, such as a zero mode waveguide, where the substrate comprises an array of zero mode waveguides housing individual complexes. In this aspect, an excitation light source is directed through a transparent substrate at an immobilized complex within a zero mode waveguide core. Due to the cross-sectional dimension of the waveguide core in the nanometer range, e.g., from about 20 to about 200 nm, the excitation light is unable to propagate through the core, and evanescent decay of the excitation light results in an illumination volume that only extends a very short distance into the core. As such, an illumination volume that contains one or a few complexes results. Thus, multiple different reactions represented in multiple waveguide cores in individual arrays can be illuminated and interrogated simultaneously. Zero mode waveguides and their application in sequencing and other analyses are described in, e.g., U.S. Pat. Nos. 6,917,726, 7,013,054, 7,181,122, 7,292,742; 7,302,146; 7,315,019 and Levene et al., Science 2003: 299:682-686 the full disclosures of which are incorporated herein by reference in their entirety for all purposes.

Other approaches to optical confinement can also be used with the methods and systems provided by the invention. For example, total internal reflectance fluorescence microscopy may be used to confine the illumination to near the surface of a substrate. This provides a similar confining effect as the zero mode waveguide, but does so without providing a structural confinement as well. Still other optical confinement techniques may generally be applied, such as those described in U.S. Pat. Nos. 7,033,764, 7,052,847, 7,056,661, and 7,056,676, previously incorporated herein by reference.

Because of the dimensions and density of features, e.g., waveguide cores and/or other optical confinements, on such substrates, highly multiplexed illumination and collection/detection systems that maximize the signal-to-noise ratio, e.g., by minimizing the production of and/or detection of background signal levels and autofluorescence, can be of beneficial use in analyzing fluorescent signals.

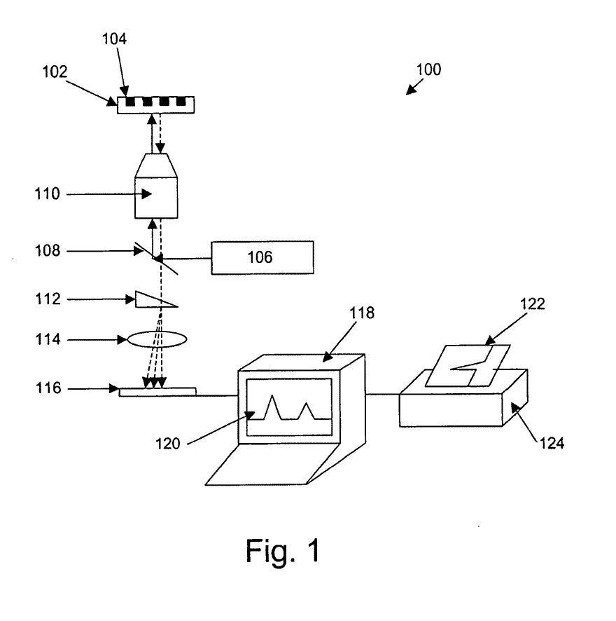

The multiplexed ZMW arrays described above are typically interrogated using a fluorescence detection system that directs excitation radiation at the various reaction regions in the array and collects and records the fluorescent signals emitted from those regions. A simplified schematic illustration of these systems is shown in FIG. 1. As shown, the system 100 includes substrate 102 that includes a plurality of discrete sources of fluorescent signals, e.g., array of zero mode waveguides 104. An excitation illumination source, e.g., laser 106, is provided in the system and is positioned to direct excitation radiation at the various fluorescent signal sources. This is typically done by directing excitation radiation at or through appropriate optical components, e.g., dichroic 108 and objective lens 110 that direct the excitation radiation at substrate 102, and particularly signal sources 104. Emitted fluorescent signals from sources 104 are then collected by the optical components, e.g., objective 110, and passed through additional optical elements, e.g., dichroic 108, prism 112 and lens 114, until they are directed to and impinge upon an optical detection system, e.g., detector array 116. The signals are then detected by detector array 116, and the data from that detection is transmitted to an appropriate data processing unit, e.g., computer 118, where the data is subjected to interpretation, analysis, and ultimately presented in a user ready format, e.g., on display 120, or printout 122, from printer 124.

While the ability to multiplex is theoretically only limited by the amount of area in which you can place your multiple samples and then analyze them, realistic analytical systems face constraints of laboratory space and cost. As such, the amount of multiplex that can be derived in the analysis of discrete fluorescent signal sources or sample regions using a realistic instrumentation system, e.g., an array of ZMWs, is somewhat limited by the ability to obtain useful signal information from increasingly small amounts of materials or small areas of substrates, plates or other analysis regions. In particular, as such signal sources are reduced in size, area or number of molecules to be analyzed, the amount of detectable signal likewise decreases, as does the signal to noise ratio of the system, e.g., due to autofluorescence background noise.

With respect to the exemplary sequencing systems described above, sources of autofluorescence background noise can typically include the components of the optical train through which the excitation radiation is directed, including the objective lens 110 or lenses, the dichroic filter(s) 108, and any other optical components, i.e., filters, lenses, etc., through which the excitation radiation passes. Also contributing to this autofluorescence background noise are components of the substrate upon which the monitored sequencing reactions are occurring, which, in the case of zero mode waveguide arrays for example, include the underlying transparent substrate that is typically comprised of glass, quartz or fused silica, as well as the cladding layer that is disposed upon the substrate, typically a metal layer such as aluminum.

In general, the present invention provides both preventive and remedial approaches to reducing impacts of autofluorescence background noise, in the context of analyses that employ illuminated reactions, e.g., multiplexed illuminated reactions. Restated, in a first general preventive aspect, the invention is directed to processes and systems that have a reduced level of autofluorescence background noise that is created and that might be ultimately detected by the system. In the additional or alternative remedial aspects, the invention provides methods and systems in which any autofluorescence background noise that is created, is filtered, blocked or masked substantially or in part from detection by the system. As will be appreciated, in many cases, both preventative and remedial approaches may be used in combination to reduce autofluorescence background noise.

### II Preventive Measures

In a first aspect, the present invention reduces the level of autofluorescence background noise generation by preventing or reducing the production of that background noise in the first instance. In particular, this aspect of the invention is directed to providing illumination of the optical signal source or sources in a way that reduces or minimizes the generation of such autofluorescence background noise.

In accordance with one aspect of the invention, the reduction in autofluorescence creation is accomplished by reducing the amount of illumination input into the system and/or directed at the substrate, e.g., by providing highly targeted illumination of only the locations that are desired to be illuminated, and preventing illumination elsewhere in the array or system. By using highly targeted illumination, one simultaneously reduces the area of the substrate that might give rise to autofluorescence, and reduces the overall amount of input illumination radiation required to be input into the system, as such input illumination is more efficiently applied.

In particular, the amount of illumination power required to be applied to the system increases with the number of signal sources that are required to be illuminated. For example, in a zero mode waveguide array that is configured in a gridded format of rows and/or columns of waveguides, multiple waveguides are generally illuminated using a linear illumination format (See, e.g., International Patent Application Nos. US2007/003570 and US2007/003804, which are incorporated herein by reference in their entirety for all purposes). Multiple rows and/or columns are then illuminated with multiple illumination lines. While linear beam spot illumination can be effective for illuminating multiple discrete regions on a substrate, e.g., multiple signal sources that are disposed in a line, there are certain deficiencies associated with this method, including excessive illumination, inefficient illumination power usage, and excessive autofluorescence background noise.

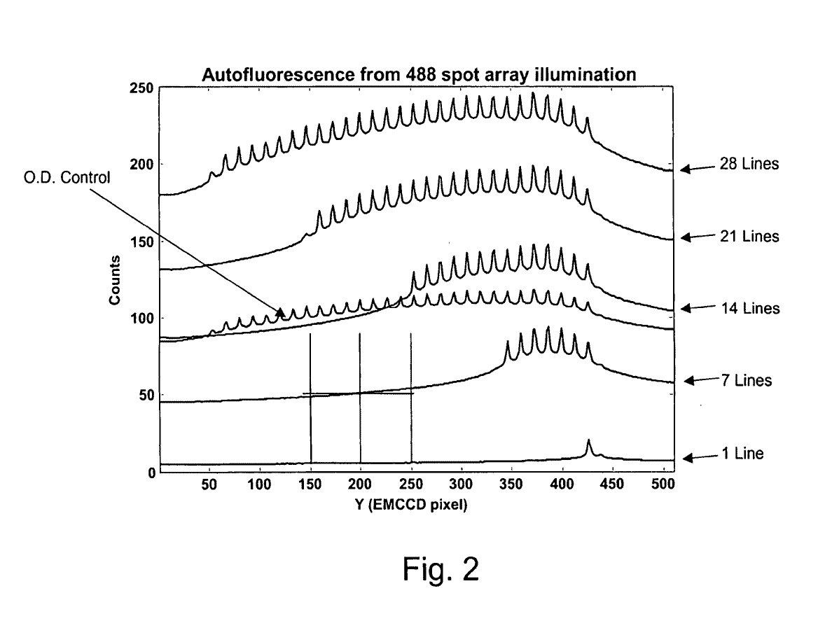

As shown in FIG. 2, as the number of illumination lines increases, it results in a linear increase in the amount of autofluorescence emanating from the system. In particular, FIG. 2 shows a plot of fluorescent signals emanating from a spotted array of Alexa488 fluorescent dye spots on a fused silica slide. As can be seen, as more illumination lines are applied to the array, the baseline fluorescence level attributable to autofluorescence background noise increases linearly with the number of illumination lines. Further, it has been demonstrated that this autofluorescence background noise derives not only from the substrate, but also from the other optical components of the system, such as the objective lens and dichroic filter(s).

Accordingly, in a first aspect, the invention reduces the amount of autofluorescence background noise by reducing the amount of excitation illumination put into the system, while still producing the desired fluorescent signals. In general, providing the same or similar levels of excitation illumination at desired locations, e.g., on the substrate, while reducing overall applied excitation illumination in the system, is accomplished through more efficient use of applied illumination by targeting that illumination only to the desired locations. In particular, by targeting illumination only at the relevant locations, e.g., primarily at only the waveguides on an array, one can reduce the amount of power required to be directed into the system to accomplish the desired level of illumination and at the substrate, yielding a consequent reduction in the amount of autofluorescence background noise that is generated at either of the substrate or those optical components through which such illumination power is directed. Additionally, because less of the substrate is being illuminated by virtue of the targeted nature of the illumination, less of the substrate will be capable of contributing to the autofluorescence background noise.

By targeted illumination or targeted illumination pattern, in accordance with the foregoing, is meant that the illumination directed at the substrate is primarily incident upon the desired locations, rather than other portions, e.g., of a substrate. For example, as alluded to above, where one desires to interrogate a number of discrete locations on a substrate for fluorescent signals, using targeted illumination would include directing discrete illumination spots at each of a plurality of the different discrete locations. Such targeted illumination is in contrast to illumination patterns that illuminate multiple locations with a single illumination spot or line, in flood or linear illumination profiles. Again, as noted above, targeting illumination provides the cumulative benefits of reducing the required amount of illumination input into the system, and illuminating less area of the substrate, both of which contribute to the problem of autofluorescence background noise.

In particular, targeted illumination, as used herein, can be defined from a number of approaches. For example, in a first aspect, a targeted illumination pattern refers to a pattern of illuminating a plurality of discrete signal sources, reaction regions or the like, with a plurality of discrete illumination spots. While such targeted illumination may include ratios of illumination spots to discrete signal sources that are less than 1, i.e., 0.1, 0.25, or 0.5 (corresponding to one illumination spot for 10 signal sources, 4 signal sources and 2 signal sources, respectively) in particularly preferred aspects, the ratio will be 1 (e.g., one spot for one signal source, i.e., a waveguide).

In accordance with preferred aspects of the present invention, optical systems that can be used with the methods, processes and systems of analyzing fluorescent materials with reduced autofluorescence can separately illuminate large numbers of discrete regions on a substrate or discrete signal sources. As used herein, separate illumination of discrete regions or locations refers to multiple individual illumination spots that are separate from each other at least the resolution of optical microscopy. In particular embodiments, the optical systems, e.g., that can be used with the methods and systems of analyzing fluorescent materials with reduced autofluorescence described herein, provide the further advantage of providing such separate illumination of densely arrayed or arranged discrete regions. Such illumination patterns can provide discrete illumination spots at a density of on the order of at least 1000 discrete illumination spots per mm², preferably at least 10,000 discrete illumination spots per mm², and in some cases, greater than 100,000 discrete illumination spots per mm², or even 250,000 discrete illumination spots per mm² or more. As will be appreciated, the foregoing illumination pattern densities will typically result in intra-spot spacing upon an illuminated substrate (in the closest dimension), of less than 100 μm, center to center, preferably, less than 20 μm, more preferably, less than 10 μm, and in many preferred cases, spacing between spots of 1 μm or less, center to center. As noted previously, such spacing generally refers to inter-spot spacing in the closes dimension, and does not necessarily reflect inter-row spacing that may be substantially greater, due to the allowed spacing for spectral separation of adjacent rows, as discussed elsewhere herein.

The optical systems that can be used with the methods and systems of the of analyzing fluorescent materials with reduced autofluorescence are generally capable of separately illuminating 100 or more discrete regions on a substrate, preferably greater than 500 discrete regions, more preferably greater than 1000 discrete regions, and still more preferably, greater than 5000 or more discrete regions. Further, such high number multiplex optics will preferably operate at the densities described above, e.g., from densities of about 1000 to about 1,000,000 discrete illumination spots per mm².

The optical systems that can be used with the methods, processes, and systems for analyzing fluorescent materials with reduced autofluorescence can provide illumination targets on the substrate that are regularly arranged over the substrate to be analyzed, e.g., provided in one or more columns and/or rows in a gridded array. Such regularly oriented target regions provide simplicity in production of the optical elements used in the optical systems. Notwithstanding the foregoing, in many cases, the optical systems that can be used in methods and systems to analyze fluorescent materials with reduced autofluorescence may be configured to direct excitation illumination in any of a variety of regular or irregular illumination patterns on the substrate. For example, in some cases, it may be desirable to target illumination at a plurality of regions that are arranged over the substrate in a non-repeating irregular spatial orientation. Accordingly, having identified such arrangement one could provide multiplex optics that direct excitation light accordingly. Likewise, such optics can readily provide for targeted illumination of rows or columns of signal sources that are disposed at irregular intra or inter-row (or column) spacings or pitches, e.g., where spots within a row are more closely spaced than spots in adjacent rows.

In the context of the multiplex optical systems that can be used with the methods and systems for analyzing fluorescent materials with reduced autofluorescence, such targeted illumination also typically refers to the direction of illumination to multiple discrete regions on the substrate, which regions preferably do not overlap to any substantial level. As will be appreciated, such targeted illumination preferably directs a large number of discrete illumination beams to a large number of substantially discrete locations on a substrate, in order to separately interrogate such discrete regions. As will also be appreciated the systems of the invention do not necessarily require a complete absence of overlap between adjacent illumination regions, but may include only a substantial lack of overlap, e.g., less than 20%, preferably less than 10% overlap and more preferably less than 5% of the illumination in one spot will overlap with an adjacent spot (when plotted as spot illumination intensity, e.g., from an imaging detector such as a CCD or EMCCD).

In still other aspects, the multiplex optics that can be used with the methods and systems of analyzing fluorescent materials with reduced autofluorescence can optionally direct in-focus illumination in a three dimensional space, thus allowing the systems of the invention to illuminate and detect signals from three dimensional substrates. Such substrates may include solid tissue samples, encased samples, bundles, layers or stacks of substrates, e.g., capillaries, planar arrays, or multilayer microfluidic devices, and the like.

A variety of components may be used to provide large numbers of illumination spots from a few, or a single illumination beam. As discussed in greater detail below, the multiplex optical element can comprise one, two, three, four or more discrete optical elements that work in conjunction to provide the desired level of multiplex as well as provide controllability of the direction of the multiplexed beams. For example and as discussed in greater detail below, one may use two or more diffraction gratings to first split a beam into a plurality of beams that will provide a plurality of collinear spots arrayed in a first dimension. Each of these beams may then be subjected to additional manipulation to provide a desired targeted illumination pattern. For example, each resulting beam may be passed through appropriate linearization optics, such as a cylindrical lens, to expand each collinear spot into an illumination line oriented orthogonal to the axis of the original series of spots. The result is the generation of a series of parallel illumination lines that may be directed at the substrate. Alternatively and preferably in some cases, the series of beams resulting from the first diffraction grating can be passed through a second diffraction grating that is rotated at a 90 degree angle (or other appropriate angle) to the first diffraction grating to provide a two dimensional array of illumination beams/spots, i.e., splitting each of the collinear spots into an orthogonally oriented series of collinear spots. In particular, if one provides a diffraction grating that provides equal amplitude to the different orders, and illuminates it with a laser beam, it will result in a row of illuminated spots, corresponding to discrete beams each traveling at a unique angle after they impinge on the grating. If a second similar grating is placed adjacent to the first but rotated by 90 degrees, it will provide a 2 dimensional grid of beamlets, each traveling with a unique angle. If the 2 gratings are identical, a square grid will result, but if the 2 gratings have different period, a rectangular grid will result. By selecting each of the diffraction gratings and the angle of rotation of the two gratings relative to each other, one can adjust spacing between and/or positioning of the columns or rows of illumination spots in the array, as desired.

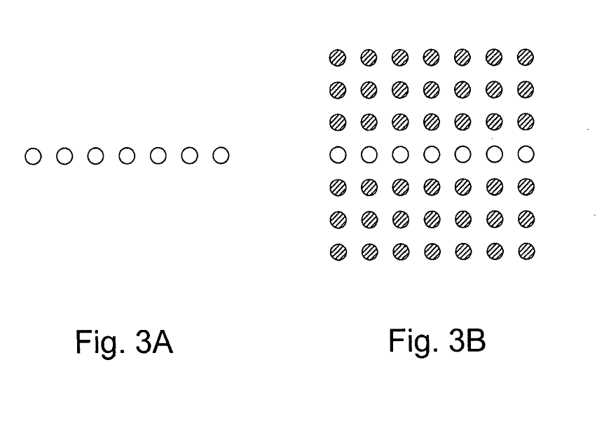

FIG. 3 provides a schematic illustration of the illumination pattern generated from a first diffraction grating, and for a first and second diffraction grating oriented 90° relative to each other. As shown, passing a single laser beam through an appropriate diffraction grating will give rise to multiple discrete beams (or “beamlets”) that are oriented in a collinear array and are represented in Panel A of FIG. 3 as a linear array of unfilled spots. By subsequently passing the linear array of beamlets through a second diffraction grating rotated orthogonally to the first, e.g., 90°, around the optical axis, one will convert each of the first set of beamlets (unfilled spots), into its own, orthogonally arrayed collinear array of beamlets (illustrated as hatched spots in Panel B of FIG. 3). The resulting set of beamlets results in a gridded array of spots, as shown in Panel B of FIG. 3.

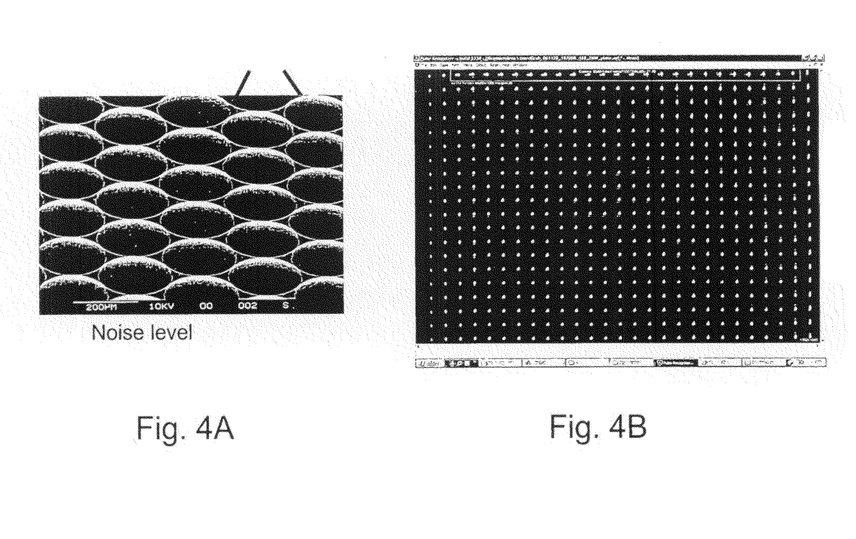

Targeting illumination to each of an array of point targets such as zero mode waveguides, can be also accomplished by a number of other methods. For example, in a first aspect, excitation radiation may be directed through a microlens array in conjunction with the objective lens, in order to generate spot illumination for each of a number of array locations. In particular, a lens array can be used that would generate a gridded array of illumination spots that would be focused upon a gridded array of signal sources, such as zero mode waveguides, on a substrate. An example of a microlens array is shown in FIG. 4, Panel A. In particular, shown is an SEM image of the array. Panel B of FIG. 4 illustrates the illumination pattern from the microlens array used in conjunction with the objective lens of the system. As will be appreciated, the lens array is fabricated so as to be able to focus illumination spots on the same pitch and position as the locations on the array that are desired to be illuminated.

In alternative and/or additional aspects, a plurality of illumination spots for targeted illumination of signal sources may be generated by passing excitation illumination through one or more diffractive optical elements (“DOE”) upstream of the objective lens. In particular, DOEs can be fabricated to provide complex illumination patterns, including arrays of large numbers of illumination spots that can, in turn, be focused upon large numbers of discrete targets.

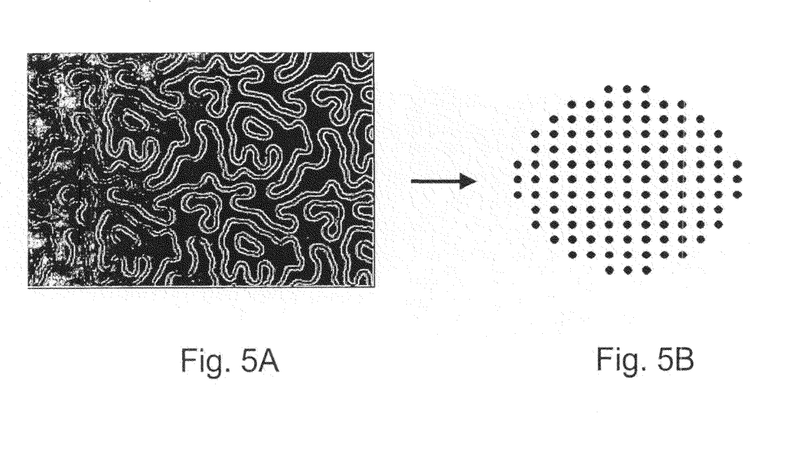

For example, as shown in FIG. 5, a DOE Phase mask, as shown in Panel A, can generate a highly targeted illumination pattern, such as that shown in Panel B, which provides targeted illumination of relatively large numbers of discrete locations on a substrate, simultaneously. In particular, the DOE equipped optical system can generally separately illuminate at least 100 discrete signal sources, e.g., zero mode waveguides, simultaneously and in a targeted illumination pattern. In preferred aspects, the DOE may be used to simultaneously illuminate at least 500 discrete signal sources, and in more preferred aspects, illuminate at least 1000, at least 5000, or at least 10,000 or more discrete signal sources simultaneously, and in a targeted illumination pattern, e.g., without substantially illuminating other portions of a substrate such as the space between adjacent signal sources or preferably between adjacent illumination spots.

Several approaches can be used to design and fabricate a DOE for use in the present invention. The purpose here is to evenly divide the single laser beam into a large number of discrete new beams, e.g., up to 5000 or more new beams, each with 1/5000 of the energy of the original beam, and each of the 5000 “beamlets” traveling in a different direction. By way of example, the DOE design requirement is to evenly space the beamlets in angles (the 2 angles are referred to herein as θₓ and θ_{y}).

As will be appreciated, the DOE (and/or a Microlens Array) will divide the light into numerous beams that are propagating at unique angles. In a preferred illumination scheme the DOE is combined with the objective lens in a planned way, such that the objective lens will perform a Fourier transform on all of the beamlets. In this Fourier transform, angle information is converted into spatial information at the image plane of the objective. After the beamlets pass through the objective, each unique θₓ and θ_{y} will correspond to a unique x,y location in the image plane of the objective. The objective properties must be known in order to correctly design the DOE or microlens. The formula for the Fourier transform is given by:

(x,y)=EFL×Tangent(θₓ,θ_{y}),

where EFL is the Effective Focal Length of the objective.

There are several different approaches to producing a DOE that will meet the needs of a fluorescence detection system that can be used with the methods and systems for reducing autofluorescence background noise. For example, one approach is through the use of a phase mask that is pixilated such that each pixel will retard the incident photons by a programmed amount. This phase retardation can again be achieved in different ways. For example, one preferred approach uses thickness of the glass element. For example, the phase mask might include a ½ inch square piece of SiO₂. Material is etched away from the top surface of the SiO₂ plate to, e.g., 64 different etch depths. This is referred to as a 64-level gray scale pattern. The final phase mask then is comprised of a pixilated grid where each pixel is etched to a particular depth. The range of etch depths corresponds to a full 2π of phase difference. Restated, a photon which impinges on a pixel with the minimum etch depth (no etching) will experience exactly 2π (additional phase evolution compared to a photon which strikes a maximum etch depth (thinnest part of the SiO₂). The pixilated pattern etched into the DOE is repeated periodically, with the result that the lateral position of the laser beam impinging on the mask is unimportant.

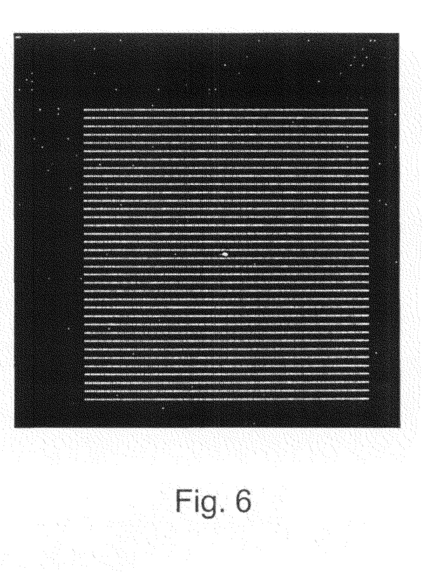

FIG. 6 shows an illumination pattern generated from a DOE that provides an array of 5112 discrete illumination spots. The DOE is configured such that the illumination spots are on a period that, when focused upon the substrate appropriately, will correspond to a discrete signal source in an arrayed substrate, e.g., a zero mode waveguide array.

In some cases, it may be desirable to provide illumination patterns that have a higher density of illumination spots than may be provided using a single DOE. In particular, the period size or spacing between adjacent illumination spots resulting from a DOE is a function of the minimum spot size of the originating illumination beam. As such, in order to obtain a higher density or smaller period size, for the illumination pattern, one may be required to employ an originating beam spot size that is smaller than desired, resulting in incomplete illumination of a desired target or enhanced difficulty in targeting a small spot to a small target. For example, in many cases, the originating beam size typically must be at least twice the period size between two adjacent resulting illumination spots from a DOE. However, where one desires an illumination spot of a larger size, the period is consequently increased.

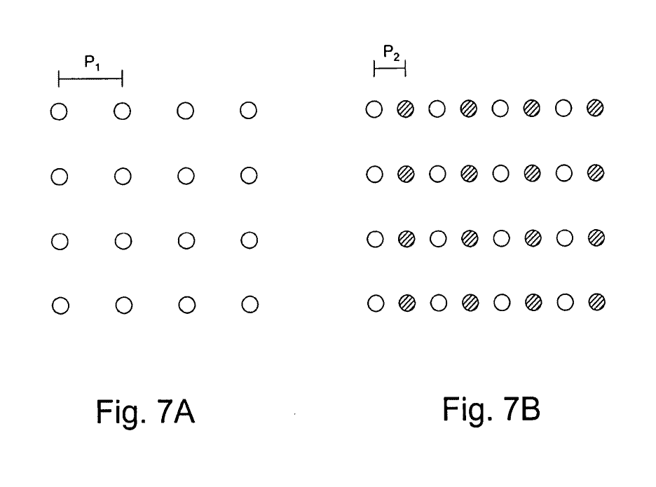

In addressing this issue, one particularly preferred approach is to utilize multiple multiplex optical elements in parallel (rather than in series). In particular, one may use two or more similar or identical DOEs in an illumination path where each DOE results in illumination spots at a period size that is twice that desired in one or more dimensions, but where each of which provides an illumination spot size that is desired. By way of example, an originating beam is first split into two identical beams using, e.g., a 50% beam splitter. Each beam is then directed through its own copy of the DOE, and the resulting multiplexed beams are imaged one half a period off from each other. As a result, the period size of the illumination spots is half that obtained with a single DOE. FIG. 7 provides a schematic illustration of the resulting illumination pattern when the illumination pattern (unfilled spots) from a first DOE having a first period P₁ (shown in panel A) and a second DOE having the same illumination pattern period P₁ (hatched spots) are overlaid as a single projection (shown in Panel B) having a new effective period P₂. As alluded to above, two, three, four or more DOEs may be used in parallel and their resulting spots overlaid, to provide different spot spacing regardless of the originating illumination spot size, providing spacing is maintained sufficient to avoid undesirable levels of spot overlap at the target locations. In addition, and as apparent in FIG. 7, by overlaying multiple illumination patterns, one can provide different spacing of illumination spots in one dimension while preserving the larger spacing. In particular, one can provide more densely arrayed illumination spots in rows while preserving a larger intra-row spacing. Such spacing is particularly useful where one wishes to preserve at least one dimension of larger spacing to account for spectral separation of signals emanating from each illuminated region. Such spacing is discussed in detail in, e.g., U.S. patent application Ser. Nos. 11/704,689, filed Feb. 9, 2007, 11/483,413, filed Jul. 7, 2006, and 11/704,733, filed Feb. 9, 2007, the full disclosures of which are incorporated herein by reference in their entirety for all purpose.

In addition to the foregoing considerations, and as will be appreciated, the actual phase evolution for the DOE is a function of the optical wavelength of the light being transmitted through it, so DOE devices will generally be provided for a specific wavelength of excitation illumination. As such, for applications in which broad spectrum or multispectral illumination is desired, the optical systems used in the methods and processes for analyzing fluorescent materials with reduced autofluorescence will typically include multiple multiplex elements, e.g., DOEs. For example, in the case of multispectral fluorescent analysis, different fluorescent dyes are typically excited at different wavelengths. As such, multiple different excitation light sources, e.g., lasers are used, e.g., one for each peak excitation spectrum of a dye. In such cases, a different multiplex element would preferably be provided for each illumination source. In the case of systems employing DOEs as the multiplex component for example, the optical path leading from each different laser would be equipped with its own DOE tailored or selected for that laser's spectrum. Accordingly, the optical systems that can be used with the methods and systems to analyze fluorescent materials with reduced autofluorescence will typically include at least one multiplex component, preferably, two, three or in many cases four or more different multiplex components to correspond to the at least one, preferably two, three, four or more different excitation light sources of varying illumination spectra.

In addition to accounting for variation in the excitation wavelength in the selection of the DOEs, the need for high-density discrete illumination may also impact the DOE specifications. In particular, as will be appreciated, because adjacent beamlets or spots may be either perfectly in or out of phase with each other, any overlap between adjacent spots on a surface may be constructive, i.e., additive, or destructive, i.e., subtractive. As such, in particularly preferred aspects where it is desired that optical systems used with the methods and systems for reducing autofluorescence provide uniform illumination of spots across the field of illumination spots, spots must be substantially separated with little or no overlap within the desired illumination region.

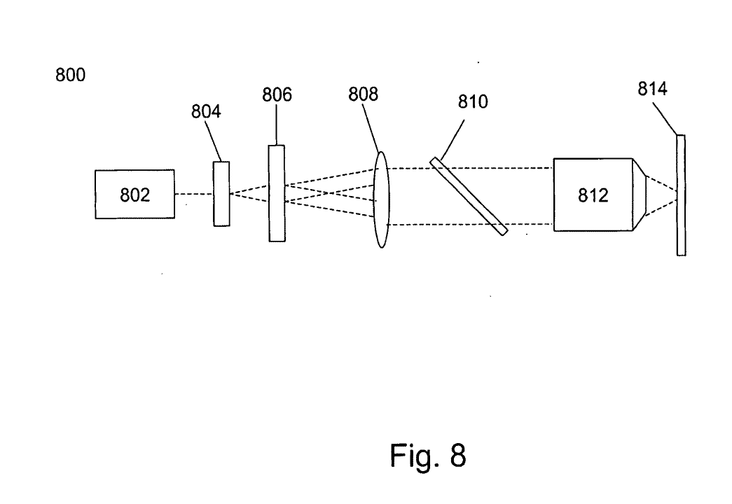

In alternative embodiments, optical systems used with the methods and systems for reducing autofluorescence, in conjunction with the multiple DOE approach described above, can employ a polarization splitter to divide the originating beam into two or more separate beams of differing polarization. Each different beam may then be split into multiple beamlets that may be overlaid in closer proximity or with greater overlap without concern for destructive interference in the overlapping regions. While a conventional polarizing beam splitter may be used to divide the originating beam, in preferred aspects, a Wollaston prism may be employed. Wollaston prisms provide for a slightly different deviation angle for s and p polarizations, resulting in the generation of two closely spaced beamlets that may be directed through the same or multiple DOEs without concern for interference from overlapping beamlets. In addition to avoiding an interference issue, the use of the Wollaston prism provides additional control of the intra-illumination spot spacing. In particular, by rotating the prism, one can adjust the spacing between grids of beamlets generated from passing the two or more different polar beam components through the DOE(s). An example of an illumination optical path including this configuration is illustrated in FIG. 8. For ease of discussion, the fluorescence path is omitted from FIG. 8. As shown, the illumination path 800 includes excitation light source 802. The excitation light is directed through polarizing splitter such as Wollaston prism 804, which splits the originating beam into its polar p and s components. Each polar beam is then passed through a multiplex component, such as one or more DOEs 804. These doubled multiplexed beams are then passed through lens 806, dichroic 810 and objective 812, to be focused as an array of illumination spots on substrate 814. As with FIG. 7, the array of illumination spots comprise overlaid patterns separated by the separation imparted by the Wollaston prism 804. Further, by rotating the prism 804, one can modulate the separation between the overlaid polar illumination patterns to adjust intra-spot spacing.

As noted above, in some cases, it may be desirable to direct excitation illumination at targets that exist in three-dimensional space, as opposed to merely on a planar substrate. In such cases, DOEs may be readily designed to convert an originating beam into an array of beamlets with different focal planes, so as to provide for three dimensional illumination and interrogation of three dimensional substrates, such as layered fluidic structures (See, U.S. Pat. No. 6,857,449) capillary bundles, or other solid structures that would be subjected to illuminated analysis.

For many applications the desired intensity of the different beamlets provided by optical systems used with the methods and systems to analyze fluorescent materials with reduced autofluorescence could be variable. For example, it may be advantageous to prescribe a varying pattern of intensities to provide a variable range of intensities that can be sampled by a grid of sample regions. Or, the desired intensity could be selected in real time by moving the sample to a beamlet of the desired intensity. Or, the grid of variable intensities could be in a repeating pattern such that a grid of sample regions with the periodicity of the repeating pattern, and the intensity of the entire grid can be selected by moving to the desired location. More importantly, variations in optical throughput can be compensated by programming the beamlet intensity. In most optical systems light near the edges of the field-of-view is vignetted such that the optical transmission is maximum at the center and falls off slowly as the observation point moves away from the center. In a typical system based on an objective lens, the vignetting may cause up to or even more than 10% lower throughput at the edge of the optical field, as compared to the center of the field. In this case, the DOE beamlet intensity pattern can be pre-programmed to accommodate such variations, e.g., to be 10% higher at the edge of the field than the center, and to vary smoothly according to the vignetting. More complicated variations in throughput can also exist in particular optical systems, and can be pre-compensated in the DOE design. For a discussion on the design of DOE phase masks, see, e.g., “Digital Diffractive optics” by Bernard Kress and Patrick Meyrueis, Wiley 2000.

Accordingly, one may provide DOEs that present multiplexed beamlets that have ranges of different powers or intensities depending upon the desired application and/or system used. In particular, the DOE may be designed and configured to present beamlets that differ in their respective power levels. As such, at least two beamlets presented will typically have different power levels, and in some cases larger subsets (e.g., 10 or more beamlets), or all of the presented beamlets may be at different power levels as a result of configuration of the DOE. Restated, a DOE can generate beamlets having power profiles to fit a given application, e.g., correcting for optical aberrations such as vignetting, providing a range of illumination intensities across a substrate, and the like. The resulting beamlets may fall within two, 5, 10, 20 or more different power profiles.

When the DOE beamlet pattern is used in combination with a microscope objective lens, the size of the individual beamlets can be modified as desired by 1) adjusting the diameter of the beam into the DOE and 2) defocusing the pattern slightly. In the case of 1) the size of the beamlet is a function of the size of the input beam, and increasing the input beam size will increase the beamlet size. In any case the final beamlet size at the ZMW plane obeys the diffraction limit, which is affected by the aperture size, and changing the input beam diameter is equivalent to changing the aperture related to the optical diffraction limit. In the case of defocusing the entire pattern, the diffraction limit is no longer obeyed but the beamlets can be made to have larger size than the diffraction limit. Further, the beamlets need not be circular—they could be elliptical by either starting with an elliptical beam input into the DOE or by defocusing the pattern in one or both dimensions. See, e.g., “Principles of Optics” by Born and Wolf, Wiley, 2006 edition.

Alternative multiplex optics systems for converting a single illumination source into multiple targeted illumination beams, e.g., to reduce autofluorescence background noise, includes, for example, fiber optic approaches, where excitation light is directed through multiple discrete optical fibers that are, in turn directed at the substrate, e.g., through the remainder of the optical train, e.g., the objective. In such context, the fiber bundles are positioned to deliver excitation illumination in accordance with a desired pattern, such as a gridded array of illumination spots.

In addition to multiplex optics that convert a single illumination beam into multiple discrete beams, as described above, certain aspects of the optical systems that can be used with the present invention may employ multiplexed illumination sources in place of a single illumination source with a separate multiplex optic component to split the illumination into multiple beamlets. Such optical systems are particularly useful in combination with the spatial filters described in greater detail below, and include, for example, arrayed solid state illumination sources, such as LEDs, diode lasers, and the like.

Alternatively, as a goal of targeted illumination in the context of the present invention is to reduce autofluorescence from excessive illumination, targeted illumination denotes illumination where a substantial percentage of the illumination that is incident upon the substrate is incident upon the desired signal source(s) as opposed to being incident on other portions of the substrate. Accounting for the often small size of signal sources, e.g., in the case of nanoscale zero mode waveguides, as well as the tolerance in direction of illumination by optical systems, such targeted illumination will typically result in at least 5% of the illumination incident upon the overall substrate being incident upon the discrete signal sources themselves. This corresponds to 95% or less wasted illumination that is incident elsewhere. In preferred aspects, that percentage is improved such at least 10%, 20% or in highly targeted illumination patterns, at least 50% of the illumination incident upon the substrate is incident upon the discrete signal sources. Conversely, the amount of illumination incident upon other portions of the substrate is less than 90%, less than 80% or in highly targeted aspects, less than 50%. Determination of this percentage is typically a routine matter of dividing the area of a substrate that is occupied by the relevant signal discrete source divided by the area of total illumination, multiplied by 100, where a region is deemed “illuminated” for purposes of this determination if it exceeds a threshold level of detectable illumination from the illumination source, e.g., 5% of that at the maximum point of a given illumination spot on the same substrate.

In still a further aspect, targeted illumination may be identified through the amount of laser power required to illuminate discrete signal sources vs. illuminating such signal sources using a single flooding illumination profile, e.g., that simultaneously illuminates an entire area in which the plurality of discrete sources is located, as well as the space between such sources. Preferably, the efficiency in targeted illumination over such flood illumination will result in the use of 20% less laser power, preferably 30% less laser power, more preferably more than 50% less laser power, and in some cases more than 75%, 90% or even 99% less laser power to achieve the same illumination intensity at the desired locations, e.g., the signal sources. As will be appreciated, the smaller the discrete illumination spot size, e.g., the more targeted the illumination, the greater the susceptibility of the system to alignment and drift issues, and calibration efforts will need to be increased.

In addition to the advantages of reduced autofluorescence, as set forth above, targeted illumination also provides benefits in terms of reduced laser power input into the system which consequently reduces the level of laser induced heating of reaction regions.

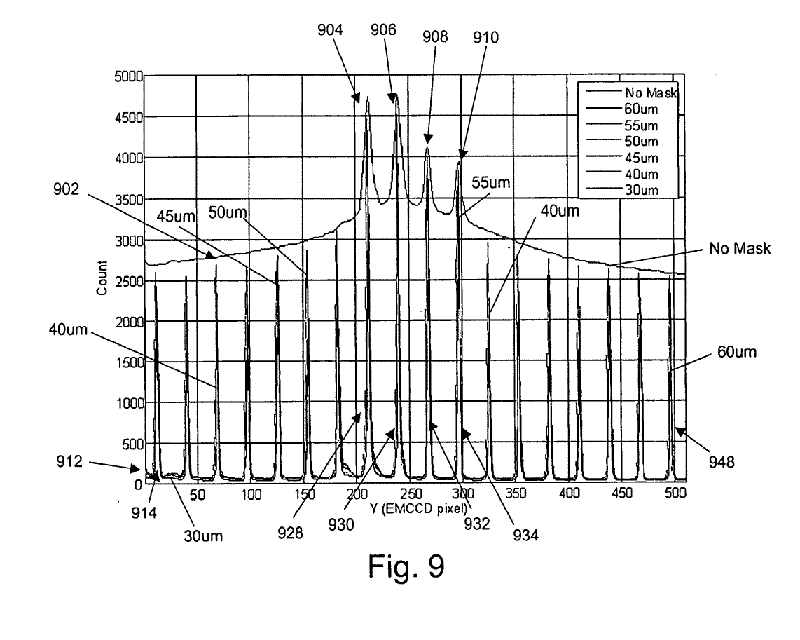

In another preventive approach, an overall optical system or one or more components through which the excitation illumination passes, may be treated to reduce the amount of autofluorescence background noise generated by the system components. By way of example, in an overall optical system, e.g., as schematically illustrated in FIG. 1, illumination may be applied to the system that results in a photobleaching of some or all of the elements of the various components that are fluorescing under normal illumination conditions. Typically, this will require an elevated illumination level relative to the normal analytical illumination conditions of the system. Photobleaching of the optical components may be carried out by exposing the optical train to illumination that is greater in one or both of intensity or power and duration. Either or both of these parameters may be from 2×, 5× 10× or even greater than that employed under conventional analysis conditions. For example, exposure of the optical train to the excitation illumination for a prolonged period, e.g., greater than 10 minutes, preferably greater than 20 minutes, more preferably greater than 50 minutes, and in some cases greater than 200 or even 500 minutes, can yield substantial decreases in autofluorescence background noise emanating from the system components. In one particular exemplary application, a 20 mW, 488 nm laser can be used to illuminate the overall system for upwards of 20 hours in order to significantly reduce autofluorescence from the components of such system. FIG. 9 shows a plot of autofluorescence counts in a system illuminated with a 20 mW 488 nm laser, following exposure of the optical train to ‘burn in’ illumination from a 7.5 mW laser at 488 nm from 0 to 1000 minutes, followed by illumination from a 162 mW laser at 488 nm from 1000 to 4600 minutes. Alternatively or additionally, other illumination sources may be employed to photobleach the optical components, including, e.g., lasers of differing wavelengths, mercury lamps, or the like. As will be appreciated, the photobleaching of the optical components may be carried out at a targeted illumination profile, e.g., a relatively narrow wavelength range such as 488 nm laser illumination, or it may be carried out under a broader spectrum illumination, depending upon the nature of the components to be photobleached and the underlying cause of the autofluorescence.

In addition to providing large numbers of discrete beams to be directed at arrayed regions on substrates, the fluorescence detection systems that can be used with the systems and methods to reduce autofluorescence optionally include additional components that provide controlled beam-shaping functionalities, in order to present optimal illumination for a given application.

For example, in the case of systems employing lens arrays, as described previously, such lens arrays may comprise a rectangular shape that results in illumination spots that are asymmetrically shaped, e.g., elliptical. Accordingly, one may include within the illumination path, one or more relatively shallow cylindrical lenses to correct the beam shape and provide a more symmetrical spot.

In addition to the various optical components described above, a number of additional cooperating optical elements may be employed with lens arrays in order to provide finer tuning of the resulting illumination pattern emanating from the multiplex component or components of the optical systems that can be used with the methods and systems for analyzing fluorescent materials with reduced autofluorescence.

In a number of cases, it will be desirable to control, and preferably independently control the direction of individual beams or subsets of beams that have been multiplexed using the systems described herein. In particular, preferred applications of the optical systems will direct multiple beams at arrays of targets that are on a pre-selected spacing, orientation and/or pitch. However in some cases, the spacing, orientation and/or pitch of target regions may not be precisely known at the time of designing the optical path, and/or may be subject to change over time. Accordingly, in some cases it will be desired to provide for independent adjustment of the direction of individual beams, or more routinely, subsets of beams multiplexed from a single originating beam.

By way of example, in the case of arrays of discrete reaction regions, typically such reaction regions will be provided at substantially known relative locations, pitch and/or orientation. In particular, such arrays may generally be presented in a gridded format of regularly spaced columns and/or rows. However, variations in the processes used to create such arrays may result in variations in such relative location, within prescribed tolerances. This is particularly an issue where the features of such arrays are on the scale of nanometers, e.g., from 10 to 500 nm in cross section.

For example, in the case of linear illumination patterns, one may wish to adjust the inter-line spacing of the illumination pattern, e.g., to adjust for variations in the inter-row or inter-column spacing of signal sources. One particular approach involves the case where a series of parallel illumination lines is created from the linearization of a row of co-linear beamlets or spots. In particular, a collinear arrangement of illumination spots generated by passing a single illumination beam through, e.g., a diffraction grating or DOE, may be converted to a series of parallel illumination lines by directing the beams through one or more cylindrical lenses. Accordingly, by simply rotating the diffraction grating or DOE around its optical axis, one can adjust the spacing of the illumination lines emanating from the cylindrical lens(es). Such adjustment both optimizes the illumination of discrete signal sources and reduces the production of autofluorescence background noise.

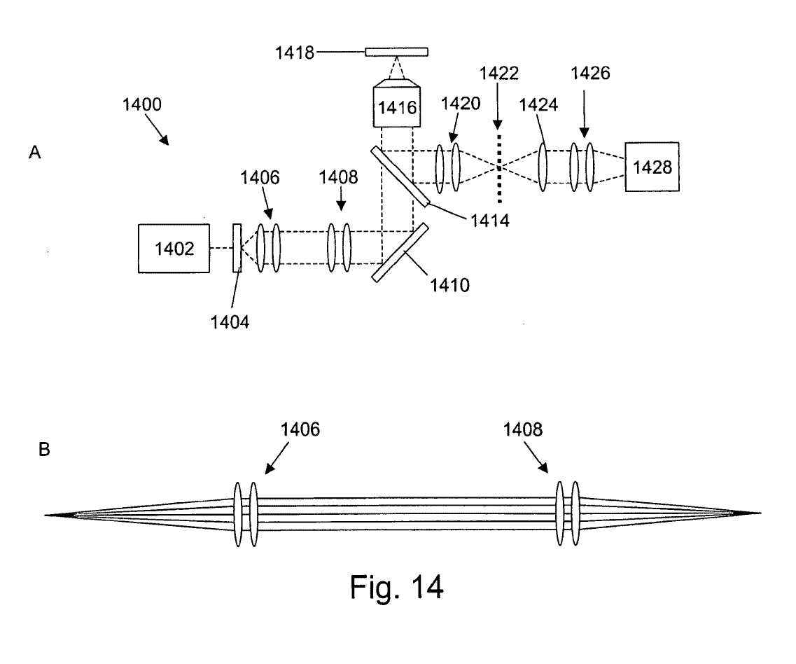

Additionally or alternatively, in some cases, it may be desirable to provide tunable lens or lenses between the multiplex component(s) and the objective of the system, in order to compensate for potential focal length variation or distortion in the objective. Such lenses may include, for example, a zoomable tube lens having a variable focal length that may be adjusted as needed. Alternatively, additional pairs of field lenses may be employed that are adjustable relative to each other, in order to provide the variable focal length. In addition to the foregoing advantages, the use of such field lenses also provide for: transformation of diverging beamlets from DOEs or other multiplex elements, into converging beamlets into the objective (as shown in FIG. 14, Panel B); provide the ability to finely adjust the angular separation of the beamlets; and provide an intermediate focusing plane so that additional elements can be incorporated, such as additional spatial filters. For example either in conjunction with field lenses as set forth above, or in some cases, in their absence, spatial filters may be applied in the illumination path. The flexibility of such an optical system can advantageously reduce the production of autofluorescence background noise while optimizing the illumination of signal sources on a substrate.

A schematic illustration of a system employing such pairs of field lenses is shown in FIG. 14. As shown, the excitation illumination source 1402 directs the originating beam through the multiplex component(s) such as DOE 1404 to create multiple beamlets. The beamlets are then passed through a pair of lenses or lens doublets, such as doublets 1406 and 1408. As noted above, the lens pair or doublet pairs 1406 and 1408, provide a number of control options over the illumination beams. For example, as shown in Panel B to FIG. 14, these doublet pairs can convert diverging beamlets into converging beamlets in advance of passing into objective 1416. Likewise, such doublet pairs may be configured to adjust the angular separation of the beamlets emanating from DOE 1404. In particular, by adjusting spacing between lenses in each doublet, one can magnify the angle of separation between beamlets. One example of this is shown in the table, below, that provides the calculated angular magnification from adjustment of spacing between lenses in each doublet of a pair of exemplary doublet lenses, e.g., corresponding to the lenses in doublets 1406 and 1408 of FIG. 14.

| Spacing | Spacing | Incoming | Outgoing | Angular |
|---|---|---|---|---|
| in First | in Second | Beamlet | Beamlet | Magnifi- |
| Doublet (mm) | Doublet (mm) | Angle (°) | Angle (°) | cation |
| 2 | 0 | 2.5505 | −2.4234 | −0.95 |
| 1 | 0 | 2.5505 | −2.48858 | −0.97 |
| 0 | 0 | 2.5505 | −2.5505 | −1.00 |
| 0 | 1 | 2.5505 | −2.6161 | −1.03 |
| 0 | 2 | 2.5505 | −2.6816 | −1.05 |

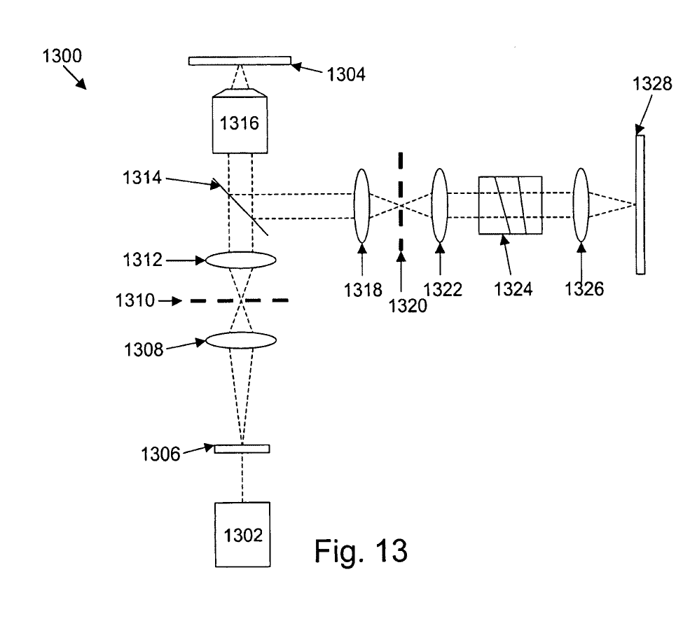

Additionally, an optional spatial filter (as shown FIG. 13 as spatial filter 1310) may be provided between the doublets 1406 and 1408, to provide modulation of the beamlets as described elsewhere herein.

The beamlets are then directed through dichroic 1414, e.g., by reflecting off optional directional mirror 1410, and through objective 1416, which focuses the illumination pattern of the beamlets onto substrate 1418. Fluorescent emissions from each discrete location that is illuminated by the discrete beamlets are then collected by the objective 1416 and reflected off dichroic 1414 to pass into the separate portion of the fluorescence path of the system. The fluorescent signals are then focused by focusing or field lens, e.g., shown as a doublet lens 1420, through a spatial filter such as confocal mask 1422, that is positioned in the focal plane of lens doublet 1420, so that only in focus fluorescence is passed. Doublet 1420 is preferably paired with objective 1416 to provide optimal image quality (both at the confocal plane and the detector image plane). The confocally filtered fluorescence is then refocused using field lens 1424 and is focused onto detector 928 using another focusing lens or lens doublet, such as doublet 1426. By providing a doublet-focusing lens, one again yields advantages of controllability as applied to the fluorescent signals, which can reduce both power usage and the generation of autofluorescence background noise.

In addition to independent adjustment of subsets of beams multiplexed from a single originating beam, it may also be desirable to independently adjust subsets of signals emanating from a substrate in response to illumination. In particular, in some cases, it may be desirable to selectively adjust certain subsets of signals in order to direct them through selected regions of the optical train, e.g., aligning with confocal masks, or to direct such signals to desired detector regions. In general, adjusting the direction of the multiple discrete fluorescent signals may be accomplished using substantially the same methods and components as those described for use in the adjustment of the excitation beams.

The use of spatial filters in the illumination path can provide a number of control advantages for the system, including dynamic and uniform control over the multiplex illumination pattern and reduction in autofluorescence background noise. In particular, one can employ a simple aperture or iris shaped or shapeable to narrow the array of beamlets that reaches the objective, and consequently the substrate. As a result, one can narrowly tailor the illumination pattern to avoid extraneous illumination of the substrate, or to target a sub-set of illumination regions or sub-region of an overall substrate. More complex spatial filters may also be employed to target different and diverse patterns of regions on the substrate by providing a mask element that permits those beamlets that correspond to the desired illumination pattern on the substrate. For example, one could target different rows and/or columns of reaction regions on an arrayed substrate, to monitor different reactions and/or different time points of similar reactions, and the like. As will be appreciated, through the use of controllable apertures, e.g., apertures that may be adjusted in situ to permit more, fewer, or different beamlets pass to the objective and ultimately the substrate, one could vary the illumination patterns dynamically to achieve a variety of desired goals.

Other types of optical elements also may be included within the illumination path. For example, in some cases, it may be desirable to include filters that modulate laser power intensity that reaches the objective. Such filters may include uniform field filters, e.g., modulating substantially all beamlets to the same extent, or they may include filters that are pixilated to different levels of a gray scale to apply adjusted modulation to different beamlets in an array. Such differential modulation may be employed to provide a gradient of illumination over a given substrate or portion of a substrate, or it may be used to correct for power variations in beamlets as a result of aberrations in the multiplex optics, or other components of the optical train, or it may be used to actively screen off or actively adjust the modulation of illumination at individual or subsets of illumination targets. As will be appreciated, LCD based filters can be employed that would provide active control on a pixel-by-pixel basis.

Any of a variety of other optical elements may similarly be included in the illumination path depending upon the desired application, including, for example, polarization filters to adjust the polarization of the illumination light reaching the substrate, scanning elements, such as galvanometers, rotating mirrors or prisms or other rastering optics such as oscillating mirrors or prisms, that may provide for highly multiplexed scanning systems, compensation optics to correct for optical aberrations of the system, e.g., vignetting, patterned spectral filters that can direct illumination light of different spectral characteristics to different portions of a given substrate, and the like. Use of such optical elements in targeting and/or polarizing the illumination light can reduce power usage and decrease autofluorescence background noise.

In particular, such spatial filter may be configured to block extraneous beamlets resulting from the diffractive orders of the multiplex components, which extraneous beamlets may contribute to noise issues. By way of example, a simple square or rectangular aperture may be provided in the illumination path after the multiplex component to permit only a limited array of beamlets to pass ultimately to the objective and substrate. Further, additional and potentially more complex spatial filters may be used to selectively illuminate portions of the substrate, which filters may be switched out in operation to alter the illumination profile and minimize the production of autofluorescence background noise. As noted above, the use of such fine-tuning optical components may be included not only in the illumination path of the system, but also in the fluorescence transmission path of the system

Although described as including various components of both an illumination path and a fluorescence path, it will be appreciated that certain aspects of the invention do not require all elements of both paths as described above. For example, in certain aspects, spectral separation of fluorescent signals may not be desirable or needed, and as such may be omitted from the systems of the invention. Likewise, in other aspects, optical signals from a substrate may not be based upon fluorescence, but may instead be based upon reflected light from the illumination source or transmitted light from the illumination source. In either case, the optical train may be configured to collect and detect such light based upon known techniques. For example, in the case of the detection of transmitted light, a light collection path may be set up that effectively duplicates the fluorescence path shown in the Figures hereto, but which is set up at a position relative to the sample opposite to that of the illumination path. Such path would typically include the objective, focusing optics, and optionally spectral filters and or confocal filters to modify the detected transmitted light, e.g., reduce scattering and autofluorescence. In such cases, dichroic filters may again, not be desired or needed.

In other preventive approaches to autofluorescence mitigation, the present invention also utilizes optical elements in the optical train or the overall system that are less susceptible to generating autofluorescence background noise. In particular, it has been determined that a substantial amount of autofluorescence from more complex optical systems derives from coatings applied to the optical components of the system, such as the coatings applied to dichroic filters and objective lenses. As a result, it will be appreciated that additional gains in the reduction of autofluorescence can be obtained through the selection of appropriate optical components, e.g., that have reduced autofluorescence. For example, in selecting an objective lens, it will typically be desirable to utilize an objective that provides a reasonably low ratio of autofluorescence to illumination, as determined on a photon count ratio. For example, in the case of a variety of objective lenses, this ratio has been determined at, e.g., 1.5×10⁻¹⁰ and 3.2×10⁻¹⁰ for Olympus model objective lenses UIS2Fluorite 60× Air objective and 40× Air Objective, respectively. Conversely, objectives that have been selected or treated to have reduced autofluorescence will typically have a ratio that is greater than this, e.g., greater than 1×10⁻¹⁰. By way of example, an Olympus model UIS1 APO 60× Air Objective provided a ratio of 6×10-11 following a photobleaching exposure as described above.

As noted above, selection of components to fall within the desired levels of autofluorescence will in many cases select for components that have fewer or no applied coating layers, or that have coating layers that are selected to have lower autofluorescence characteristics under the particular applied illumination conditions. Of particular relevance to the instant aspect is the selection of dichroic filters that have been selected to have lower autofluorescence deriving from their coatings, either through selection of coating materials or use of thinner coating layers.

### III. Prevention of Detection of Autofluorescence

In an alternative or additional aspect, the invention is directed to a remedial approach to background signal levels, e.g., that reduce the amount of background signal or autofluorescence that is detected or detectable by the system. Typically, this aspect of the invention is directed to filtering signals that are derived from the signal sources or arrays in such a way that highly relevant signals, e.g., those from the signal sources and not from irrelevant regions, are detected by the system. As will be appreciated, this aspect of the invention may be applied alone, or in combination with the preventive measures set forth above, in order to maximize the reduction of the impact of background signal levels.

In the context of one aspect of the invention, it has been determined that a large amount of the autofluorescence background noise constitutes “out of focus” fluorescence, or fluorescence that is not within the focal plane of the system when analyzing a given reaction region or regions. For example, autofluorescence that derives from the substrate portion of the overall systems of the invention, e.g., substrate 102 in FIG. 1, derives from locations in the substrate that are outside of the focal plane of the optical system. In particular, where the optical system is focused upon the back surface of the substrate, the autofluorescence that derives from the entirety of the thickness of the substrate, from the cladding layer above the back surface of the substrate, or from other points not within the focal plane of the system, will generally be out of focus. Likewise, autofluorescence from optical components of the system that are subjected to excitation illumination also are typically not within the focal plane of the instrument. Such components include, for example and with reference to FIG. 1, objective lens 110, and dichroic 108. Because these components transmit the full excitation illumination, they are more prone to emitting autofluorescence.

In order to mitigate the contribution of the out of focus components in the systems of the invention, the collected signals from each of these signal sources is subjected to a spatial filtering process whereby light noise contributions that are not within the focal plane of the optical system are minimized or eliminated.

Accordingly, in at least one aspect, the invention employs a spatial filter component to filter out autofluorescence that is out of the focal plane of the objective lens. One example of such a spatial filter includes a confocal mask or filter placed in the optical train. In particular, the fluorescent signals from the discrete regions on the substrate that are collected by the objective and transmitted through the optical train, are passed through a focusing or field lens and a confocal filter placed in the image plane of that lens. The light passed through the confocal filter is subsequently refocused and imaged onto a detector. Fluorescence that is not in the focal plane of the objective will be blocked by the confocal aperture, and as a result, will not reach the detector, and consequently will not contribute to the fluorescent noise. This typically includes scattered or reflected fluorescence, autofluorescence of substrates and other system components and the like. In the context of the present invention, the spatial filtering process is applied to the fluorescent signals from a large number of discrete signal sources, simultaneously, e.g., without the use of scanning, galvo or other rastering systems. In particular, the confocal filters applied in the systems of the invention typically include a large number of confocal apertures that correspond to the number of regions on the substrate from which signals are desired to be obtained. In accordance with array sizes as set forth elsewhere herein, for example, the confocal masks used in this context can typically include an array of at least about 100 or more discrete confocal apertures, preferably greater than 500 discrete confocal apertures, more preferably greater than 1000 discrete confocal apertures, and still more preferably, between about 1000 and about 5000 apertures, and in some cases greater than 5000 or more discrete confocal apertures. Such confocal masks will also typically be arrayed in a concordant pitch and/or alignment with the signal source arrays, so that signal from each discrete source that is desired to be observed will pass through a separate confocal aperture in the confocal mask. The actual size and spacing of the confocal pinholes will typically vary depending upon the desired illumination pattern, e.g., number and spacing of illumination beamlets, as well as the characteristics of the optical system.

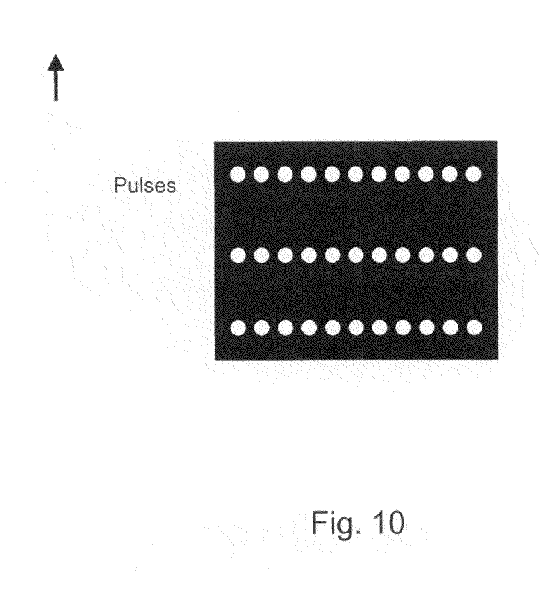

While individual pinhole apertures corresponding to individual signal sources are generally preferred, it will be appreciated that other spatial filters may also be employed that provide for simpler alignment, such as using narrow slits to reduce out of focus signal components in at least one dimension. Individual slits could be employed in filtering signals from a plurality of signal sources in a given row, column or other defined region, e.g., adjacent signal sources on the diagonal. FIG. 10 shows a schematic of a partial confocal mask showing apertures that are provided on the same pitch and arrangement as the signals being focused therethrough, e.g., corresponding to fluorescent signals imaged from an array of zero mode waveguides. As noted previously, where confocal slits, or other filters applied to multiple signal sources are used, they may number less than the total number of individual signal sources and may conform to the number of columns and or rows of signal sources, e.g., greater than 10, 20, 50 or even 100 or more confocal apertures.

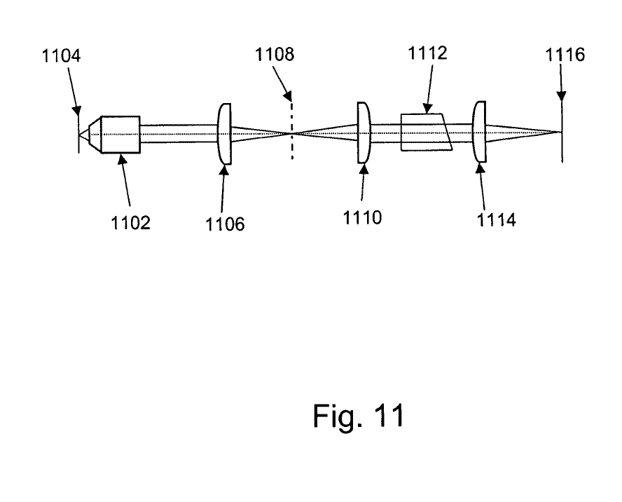

An example of an optical train including such a confocal filter is schematically illustrated in FIG. 11. As shown, an objective lens 1102 is positioned adjacent to a substrate, such as zero mode waveguide array 1104 having the reaction regions of interest disposed upon it, so as to collect signals emanating from the substrate, as well as any autofluorescence that emanates from the substrate. The collected fluorescence is then focused through a first focusing lens 1106. A confocal mask 1108 is placed in the focal plane of the first focusing lens 1106. Spatially filtered fluorescence that is passed by the confocal mask is then refocused through a second focusing lens 1110 and passed through the remainder of the optical train. As shown, this includes a wedge prism 1112 to spatially separate spectral components of the fluorescence, and third focusing lens 1114, that focuses the image of the fluorescence derived from the focal plane of the objective 1102, onto a detector, such as EMCCD 1116. By placing the confocal mask in the focal plane of the first focusing lens 1106, autofluorescence components that are out of the focal plane of the objective lens (and thus not focused by the focusing lens at the confocal mask 1108) will be blocked or filtered, and only fluorescence that is in the focal plane, e.g., fluorescent signals and any autofluorescence that exists in the focal plane, will be passed and imaged upon the detector 1116, and detected. In comparative experiments, autofluorescence background signals were reduced approximately 3 fold through the incorporation of a confocal mask, in both two and three laser systems.

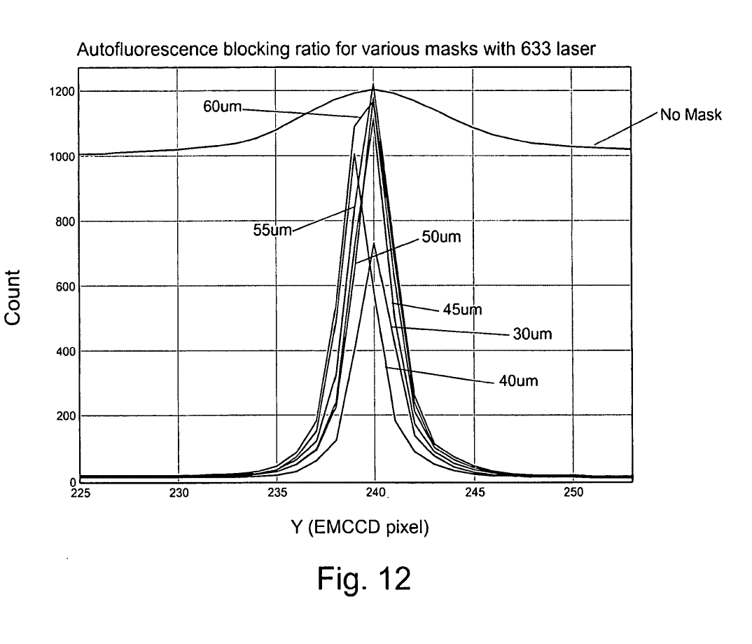

FIG. 9 provides an illustration of the effects of out of focus autofluorescence as well as the benefits of a confocal mask in reducing such autofluorescence. In particular, FIG. 9 shows a plot of autofluorescence levels as a function of the location of the image of the autofluorescence on an EMCCD detector, from a substrate that was illuminated with four illumination lines at 488 nm. As shown, the upper plot 902 corresponds to autofluorescence image from 4 illumination lines, but in the absence of a confocal mask filtering the out of focus components. The 4 peaks (904-910) correspond to the elevated autofluorescence at the illumination lines on the substrate while the baseline corresponds to the overall global autofluorescence across the remainder of the substrate. By contrast, inclusion of a confocal mask provides a substantial reduction in the amount of the out of focus autofluorescence from the system. In particular, the lower plot 912, reflects the confocally filtered traces through a number of different slit sizes, where each aggregate peak (914-948) corresponds to the position of the slits in the confocal masks used. As can be seen, peaks 928-934 correspond to the location of the illumination lines, and as such have a higher amount of in focus autofluorescence. The remaining peaks also represent autofluorescence that is in the focal plane and thus not filtered by the confocal mask. FIG. 12 shows an expanded view of the various plots with illumination at 633 nm, with the upper plot reflecting an unfiltered level of autofluorescence imaged at a given position on the detector, while the lower plots reflect the autofluorescence at the same position but filtered using confocal masks having slit sizes of 60 nm, 55 nm, 50 nm, 45 nm, 40 nm, and 30 nm. The decreasing size of the autofluorescence peak is correlated to the reduction in the dimensions of the slit in the confocal mask used.

Notwithstanding this in focus component, it can be easily seen that the provision of the confocal mask provides a significant reduction in the overall autofluorescence that is detected (as indicated by the area under each of the two plots). As noted, the confocal mask used in the example shown in FIG. 9 employed confocal slits for a linear illumination profile. It will be appreciated that alternative mask configurations may be employed as well, such as the use of arrayed pinholes in the confocal mask, in order to provide arrayed spot or targeted illumination as discussed elsewhere herein.

Other additional approaches to reduction of generated autofluorescence include spectral filtering of autofluorescence noise, through the incorporation of appropriate filters within the optical train, and particularly the collection aspects of the optical train. It has been observed that a substantial amount of autofluorescence signal in a typical illumination profile, e.g., in a wavelength range of from about 720 nm to about 1000 nm, falls within spectral ranges that do not overlap with desired detection ranges, e.g., from about 500 nm to about 720 nm. As such, elimination of at least a portion of autofluorescence noise may be accomplished by incorporating optical filters that block light outside of the desired range, e.g., long or short pass filters that block light of a wavelength greater than about 720 nm or less than about 500 nm. Such filters are generally made to order from optical component suppliers, including, e.g., Semrock, Inc., Rochester N.Y., Barr Associates, Inc., Westford, Mass., Chroma Technology Corp., Rockingham Vt.

FIG. 13 provides a general schematic for an embodiment of a fluorescence detection system comprising optical elements that can reduce both the production and detection of autofluorescence background noise. As shown, the overall system 1300 generally includes an excitation illumination source 1302. Typically, such illumination sources will comprise high intensity light sources such as lasers or other high intensity sources such as LEDs, high intensity lamps (mercury, sodium or xenon lamps), laser diodes, and the like. In preferred aspects, the sources will have a relatively narrow spectral range and will include a focused and/or collimated or coherent beam. For the foregoing reasons, particularly preferred light sources include lasers, solid-state laser diodes, and the like.

The excitation illumination source 1302 is positioned to direct light of an appropriate excitation wavelength or wavelength range, at a desired fluorescent signal source, e.g., substrate 1304, through an optical train. In accordance with the present invention, the optical train typically includes a number of elements, e.g., one or more microlens and/or one or more DOE, to appropriately direct excitation illumination at the substrate 1304, and receive and transmit emitted signals, e.g., with reduced autofluorescence background noise, from the substrate to an appropriate detection system such as detector 1328. In accordance with the present invention, the excitation illumination from illumination source 1302 is directed first through optical multiplex element (or elements 1306), e.g., one or more microlens and/or one or more DOE, to multiply the number of illumination beams or spots from an individual beam or spot from the illumination source 1302. The multiplexed beam(s) is then directed via focusing lens 108 through optional first spatial filter 1310, and focusing lens 1312. As discussed in greater detail above, spatial filter 1310 optionally provides control over the extent of multiplex beams continuing through the optical train reduces the amount of any scattered excitation light from reaching the substrate. The spatially filtered excitation light is then passed through dichroic 1314 into objective lens 1316, whereupon the excitation light is focused upon the substrate 1304. Dichroic 1314 is configured to pass light of the spectrum of the excitation illumination while reflecting light having the spectrum of the emitted signals from the substrate 1304. Because the excitation illumination is multiplexed into multiple beams, multiple discrete regions of the substrate are separately illuminated.

Fluorescent signals that are emitted from those portions of the substrate that are illuminated, are then collected through the objective lens 1316, and, because of their differing spectral characteristics, they are reflected by dichroic 1314, through focusing lens 1318, and second spatial filter, such as confocal mask 1320, and focusing lens 1322. Confocal mask 1320 is typically positioned in the focal plane of lens 1318, so that only in-focus light is passed through the confocal mask, and out-of focus light components are blocked. This results in a substantial reduction in noise levels from the system, e.g., that derive from out of focus contributors, such as autofluorescence of the substrate and other system components.

As with the excitation illumination, the signals from the multiple discrete illuminated regions on the substrate are separately passed through the optical train. The fluorescent signals that have been subjected to spatial filtering are then passed through a dispersive optical element, such as prism assembly 1324, to separately direct spectrally different fluorescent signal components, e.g., color separation, which separately directed signals are then passed through focusing lens 1326 and focused upon detector 1328, e.g., an imaging detector such as a CCD, ICCD, EMCCD or CMOS based detection element. Again, the spectrally separated components of each individual signal are separately imaged upon the detector, so that each signal from the substrate will be imaged as separate spectral components corresponding to that signal from the substrate. For a discussion of the spectral separation of discrete optical signals, see, e.g., Published U.S. Patent Application No. 2007-0036511, incorporated herein by reference in its entirety for all purposes.

As will be appreciated, a more conventional configuration that employs reflected excitation light and transmitted fluorescence may also be employed by altering the configuration of and around dichroic 1314. In particular, dichroic 1314 could be selected to be reflective of the excitation light from illumination source 1302, and transmissive to fluorescence from the substrate 1304. The various portions of the optical train are then arranged accordingly around dichroic 1314. Notwithstanding the foregoing, fluorescence reflective optical trains are particularly preferred in the applications of the systems of the invention. For a discussion on the advantages of such systems, see, e.g., U.S. patent application Ser. Nos. 11/704,689, filed Feb. 9, 2007, 11/483,413, filed Jul. 7, 2006, and 11/704,733, filed Feb. 9, 2007, the full disclosures of which are incorporated herein by reference in their entirety for all purpose.

As noted with reference to FIG. 13, the fluorescence path of the system typically includes optics for focusing the signals from the various regions onto discrete locations on a detector. As with the direction of excitation illumination onto a plurality of discrete regions on a relatively small substrate area, likewise, each of the plurality of discrete fluorescent signals is separately imaged onto discrete locations on a relatively small detector area. This is generally accomplished through focusing optics in the fluorescence path positioned between the confocal filter and the detector (optionally in combination with optical components provided with the confocal filter (see discussion below). As with the illumination path, the fluorescence path can typically direct at least 10, preferably at least 100, more preferably at least 500, or 1000 or in some cases at least 5000 discrete fluorescent signals to discrete locations on the detector. Because these detectors, e.g., EMCCDs have relatively small areas, these signals will typically be imaged at relatively high densities at the EMCCD plane. Such densities typically reflect the illumination spot density at the substrate plane divided by the relative size of image of the substrate as compared to the actual substrate size, due to magnification of the system, e.g., imaging signal sources on an area that is 3600× larger than the illumination pattern (e.g., 250,000 illumination density/3600). Although in preferred aspects, the images of the fluorescent signal components will be oriented in an array of two or more rows and/or columns of imaged signals, in order to provide the densities set forth herein, it will be appreciated that density may be determined from images arrayed in other formats, such as linear arrays, random arrays, and the like. Further, while the imaged signals of the invention will preferably number greater than 10, 100, 500, 1000 or even greater than 5000, density may be readily determined and applicable to as few as two discrete images, provided such images are sufficiently proximal to each other to fit within the density described.

The systems of fluorescence detection that can be used with the methods and systems provided herein, e.g., for reducing autofluorescence, also typically include spectral separation optics to separately direct different spectral components of the fluorescent signals emanating from each of the discrete regions or locations on the substrate, and image such spectral components onto the detector. In some cases, the image of the spectral components of a given discrete fluorescent signal will be completely separate from each other. In such cases, it will be appreciated that the density of the discrete images on the detector may be increased by the number of discrete fluorescent components. For example, where a fluorescent signal is separated into four spectral components, each of which is discretely imaged upon the detector, such density could be up to 4 times that set forth above. In preferred aspects, however, the separate direction of spectral components from a given fluorescent signal will not impinge upon completely discrete regions of the detector, e.g., image of one spectral component would impinge on overlapping portions of the detector as another spectrally distinct component (See, e.g., Published U.S. Patent Application No. 2007-0036511, U.S. patent application Ser. Nos. 11/704,689, filed Feb. 9, 2007, 11/483,413, filed Jul. 7, 2006, and 11/704,733, filed Feb. 9, 2007, the full disclosures of which are incorporated herein by reference in their entirety for all purposes.

While the separation optics may include multiple elements such as filter/mirror combinations to separately direct spectrally distinct components of each fluorescent signal, in preferred aspects, a dispersive optical element is used to separately direct the different spectral components of the fluorescent signals to different locations on the substrate.

### IV. Other Applications for the Optical Systems and Fluorescence Detection Systems Described Herein

As noted previously, the optical systems, fluorescence detection systems and the methods of their use that are described herein are broadly applicable to a wide variety of applications where it is desirable to illuminate multiple discrete regions of a substrate and obtain responsive optical signals from such regions, e.g., with reduced autofluorescence background noise. Such applications include analysis of fluorescent or other optically monitored reactions or other processes, optical interrogation of, e.g., digital optical media, spatial characterization, e.g., holography, laser driven rapid prototyping techniques, multipoint spatial analysis, e.g., for mobility/motility analysis, as well as a large number of other general uses.

In one particularly preferred example, the methods and systems of the invention are applied in the analysis of nucleic acid sequencing reactions being carried out in arrays of optically confined reaction regions, such as zero mode waveguides. In particular, the methods and systems are useful for analyzing fluorescent signals that are indicative of incorporation of nucleotides during a template dependent polymerase mediated primer extension reaction, where the fluorescent signals are not just indicative of the incorporation event but also can be indicative of the type of nucleotide incorporated, and as a result, the underlying sequence of the template nucleic acid. Such nucleic acid sequencing processes are generally referred to as “sequencing by incorporation” or sequencing by synthesis” methods, in that sequence information is determined from the incorporation of nucleotides during nascent strand synthesis. Although the systems and methods of the invention are much more broadly applicable than this preferred application, the advantages and benefits of these systems and methods are exploited to a great degree in such applications. As such, for ease of discussion, the systems and methods of the invention are described in greater detail with respect to such applications, although they will be appreciated as having much broader applicability.

Typically, in sequencing by synthesis processes, a complex of a polymerase enzyme, a target template nucleic acid sequence and a primer sequence is provided. The complex is generally immobilized via the template, the primer, the polymerase or combinations of these. When the complex comes into contact with a nucleotide that is complementary to the base in the template sequence immediately adjacent to where the primer sequence is hybridized to that template, the polymerase will typically incorporate that nucleotide into the extended primer. By associating a fluorescent label with the nucleotide, one can identify the incorporation event by virtue of the presence of the label within the complex. In most SBI applications, the incorporation event terminates primer extension by virtue of a blocked 3′ group on the newly incorporated nucleotide. This generally allows the immobilized complex to be washed to remove any non-incorporated label, and observed, to identify the presence of the label. Subsequent to identifying incorporation, the complex is typically treated to remove any terminating blocking group and/or label group from the complex so that subsequent base incorporations can be observed. In some processes, a single type of base is added to the complex at a time and whether or not it is incorporated is determined. This typically requires iterative cycling through the four bases to identify extended sequence stretches. In alternative aspects, the four different bases are differentially labeled with four different fluorescent dyes that are spectrally distinguishable, e.g., by virtue of detectably different emission spectra. This allows simultaneous interrogation of the complex with all four bases to provide for an incorporation even in each cycle, and also provide for the identification of the base that was incorporated, by virtue of its unique spectral signature from its own label. In general, such systems still typically require addition of a terminated nucleotide followed by a washing step in order to identify the incorporated nucleotide.

In another approach, nucleotide incorporation is monitored in real time by optically confining the complex such that a single molecular complex may be observed. Upon incorporation, a characteristics signal associated with incorporation of a labeled nucleotide, is observed. Further, such systems typically employ a label that is removed during the incorporation process, e.g., a label coupled to the polyphosphate chain of a nucleotide or nucleotide analog, such that additive labeling effects do not occur. In particular, such optical confinements typically provide illumination of very small volumes at or near a surface to thereby restrict the amount of reagent that is subject to illumination to at or near the complex. As a result, labeled nucleotides that are associated with the complex, e.g., during incorporation, can yield a distinct signal indicative of that association. Examples of optical confinement techniques include, for example, total internal reflection fluorescence (TIRF) microscopy, where illumination light is directed at the substrate in a manner that causes substantially all of the light to be internally reflected within the substrate except for an evanescent wave very near to the surface.

Other preferred optical confinement techniques include the use of zero mode waveguide structures as the location for the reaction of interest. Briefly, such zero mode waveguides comprise a cladding layer disposed over a transparent substrate layer with core regions disposed through the cladding layer to the transparent substrate. Because the cores have a cross-sectional dimension in the nanometer range, e.g., from about 10 to about 200 nm, they prevent propagation of certain light through the core, e.g., light that is greater than the cut-off frequency for the given cross-sectional dimension for such core. As a result, and as with the TIRF confinement, light entering the waveguide core through one or the other end, is subject to evanescent decay, that results in only a very small illumination volume at the end of the core from which the light enters.

In the context of SBI applications, immobilizing the complex at one end of the core, e.g., on the transparent substrate, allows for illumination of the very small volume that includes the immobilized complex, and thus the ability to monitor few or individual complexes. Because these systems focus upon individual molecular interactions, they typically rely upon very low levels of available signal. This in turn necessitates more sensitive detection components. Further, in interrogating large numbers of different reactions, one must apply a relatively large amount of illumination radiation to the substrate, e.g., to provide adequate illumination to multiple reaction regions. As a result, there is the potentiality for very low signal levels coming from individual molecules coupled with very high noise levels coming from highly illuminated substrates and systems and sensitive optical detection systems.

Although described primarily in terms of single molecule analysis, and particularly for sequence determination applications, the optical systems and fluorescence detection systems described herein, with their highly multiplexed confocal optics, are useful in almost any application in which one wishes to interrogate multiple samples for a fluorescent signal or signals and detect the signals with reduced autofluorescence background noise. For example, in related research fields, the systems of the invention are directly applicable to the optical interrogation of arrays of biological reactions and/or reactants. These may range from the simple embodiment of a highly multiplexed multi-well reaction plates, e.g., 96, 386 or 1536 well plates, or higher multiplexed “nanoplates”, such as the Openarray® plates from Biotrove, Inc., to the more complex systems of spotted or in-situ synthesized high density molecular or biological arrays. In particular, biological arrays typically comprise relatively high density spots or patches of molecules of interest that are interrogated with and analyzed for the ability to interact with other molecules, e.g., probes, which bear fluorescent labeling groups. Such arrays typically employ any of a variety of molecule types for which one may desire to interrogate another molecule for its interaction therewith. These may include oligonucleotide arrays, such as the Genechip® systems available from Affymetrix, Inc (Santa Clara, Calif.), protein arrays that include antibodies, antibody fragments, receptor proteins, enzymes, or the like, or any of a variety of other biologically relevant molecule systems.

In its most prolific application, array technology employs arrays of different oligonucleotide molecules that are arrayed on a surface such that different locations, spots or features have sequences that are known based upon their position on the array. The array is then interrogated with a target sequence, e.g., an unknown sample sequence that bears a fluorescent label. The identity of at least a portion of that target sequence is then determinable from the probe with which it hybridizes, which is, in turn, known or determinable from the position on the array from which the fluorescent signal emanates.

As feature sizes in arrays are reduced in order to provide greater numbers of molecules, the needs for highly multiplexed optical systems and fluorescence detection systems described herein are increased. Likewise, as array sizes increase, the demands on conventional scanning systems are further increased. As such, the systems of the invention, either as static array illumination, or as scanning or otherwise translatable systems, as described above, are particularly useful in this regard.

In commercially available systems, interrogation of large arrays of molecules has been carried out through either the use of image capture systems, or through the iterative scanning of the various spots or features of the array using, e.g., confocal scanning microscopes. The optical systems and fluorescence detection systems described herein, in contrast, provide a simultaneous, confocal examination of highly multiplexed arrays of different molecules through their discrete illumination and signal collection, e.g., signal collection with reduced autofluorescence background noise. Further, the spectroscopic aspects of the invention further enhance this functionality in the context of multi-label applications, e.g., where different targets/probes are labeled with spectrally distinguishable fluorescent labels.

The optical systems and fluorescence detection systems described herein are similarly useful in a variety of other multiplexed spectroscopic analyses. For example, in the field of microfluidic systems, large numbers of microfluidic conduits may be arrayed and analyzed using the systems of the invention. Such microfluidic systems typically comprise fluidic conduits disposed within a glass or plastic substrate, through which reagents are moved, either electrokinetically or under pressure. As reagents flow past a detection point, they are interrogated with an excitation source, e.g., a laser spot, and the fluorescence is monitored, e.g., with an increased signal-to-noise ratio. Examples of microfluidic systems include, for example, capillary array electrophoresis systems, e.g., as sold by Applied Biosystems Division of Applera, Inc., as well as monolithic systems, such ozas the LabChip® microfluidic systems available from Caliper Life Sciences, Inc. (Hopkinton, Mass.), and the Biomark™ and Topaz® systems available from Fluidigm®, Inc. (So. San Francisco, Calif.). While the fluidic conduits of these systems are predominantly arrayed in two dimensions, e.g., in a planar format, the systems of the invention may be configured to provide confocal illumination and detection from a three dimensional array of signal sources. In particular, diffractive optical elements used in certain aspects of the multiplex optics of the invention may be configured to provide illumination spots that are all in focus in a three dimensional array. Such three dimensional arrays may include multilayer microfluidic systems, bundled capillary systems, stacked multi-well reaction plates, or the like.

In addition to the foregoing, these optical systems and fluorescence detection systems described herein are similarly applicable to any of a variety of other biological analyses, including, for example, multiplexed flow cytometry systems, multiplexed in-vivo imaging, e.g., imaging large numbers of different locations on a given organism, or multiple organisms (using, e.g., infrared illumination sources, e.g., as provided in the Ivis® series of imaging products from Caliper Life Sciences, Inc.

While the primary applications for the systems of the invention are geared toward multiplexed analysis of chemical, biochemical and biological applications, it will be appreciated that the highly multiplexed systems of the invention, with their high signal to noise capability, also find use, in whole or in part, in a variety of other optical interrogation techniques. For example, the highly multiplexed confocal optics and detection methods of the invention may be readily employed in high bandwidth reading and/or writing of digital data to or from optical media. Likewise, the highly multiplexed illumination systems of the invention may be employed in optically driven tools, such as laser based rapid prototyping techniques, parallel lithography techniques, and the like, where highly multiplexed laser beams can be applied in the fabrication and/or design processes.

Although described in some detail for purposes of illustration, it will be readily appreciated that a number of variations known or appreciated by those of skill in the art may be practiced within the scope of present invention. To the extent not already expressly incorporated herein, all published references and patent documents referred to in this disclosure are incorporated herein by reference in their entirety for all purposes.

What is claimed is:

1-94. (canceled)

95. A system for monitoring a plurality of discrete fluorescent signals from a plurality of discrete fluorescent signal sources, the system comprising:
a substrate in a first focal plane having the plurality of discrete signal sources disposed thereon;
a first excitation illumination source providing light having a first spectrum;
a detector for detecting the plurality of discrete fluorescent signals from the plurality of discrete fluorescent signal sources; and,
an optical train positioned to simultaneously direct excitation illumination from the first excitation illumination source to each of the plurality of discrete fluorescent signal sources on the substrate and to direct the discrete fluorescent signals from the plurality of discrete fluorescent signal sources to the detector, wherein the optical train comprises:
an objective lens in the first focal plane, which objective lens is focused at the substrate and collects the discrete fluorescent signals from the plurality of discrete fluorescent signal sources on the substrate,
a first pair of tunable lenses that are adjustable relative to each other, and
a first diffractive optical element (DOE) configured to convert a single originating illumination beam from the first excitation illumination source into a plurality of discrete illumination beams, each beam being directed at a different one of the plurality of discrete fluorescent signal sources on the substrate.

96. The system of claim 95, wherein the substrate comprises first and second opposing surfaces, the first surface being more proximal to the optical train than the second surface, and the first focal plane being substantially coplanar with the second surface.

97. The system of claim 95, wherein the plurality of discrete fluorescent signal sources on the substrate are at a density of greater than 1000, greater than 10,000, or greater than 250,000 discrete florescent signal sources per mm².

98. The system of claim 95, wherein the objective lens has a ratio of excitation illumination to autofluorescence of greater than 1×10⁻¹⁰.

99. The system of claim 95, wherein the first pair of tunable lenses is positioned to do at least one of the following: a) transform diverging beamlets from the first DOE into converging beamlets into the objective lens, b) finely adjust the angular separation of the converging beamlets, c) adjust the focal length of an illumination path created by directing the excitation illumination from the first excitation illumination source to each of the plurality of discrete fluorescent signal sources, d) control spacing between beams in the plurality of discrete illumination beams, and e) provide an intermediate focusing plane into which at least one additional optical element can be incorporated.

100. The system of claim 99, wherein the first pair of tunable lenses is positioned to provide an intermediate focusing plane into which at least one additional optical element can be incorporated, and wherein the at least one additional optical element comprises a confocal filter comprising a plurality of discrete confocal apertures, each of the apertures being oriented to pass in-focus light from a different one of the discrete fluorescent signal sources onto a different location on the detector.

101. The system of claim 100, wherein the confocal filter comprises at least 10 discrete confocal apertures positioned in a focal plane of an image of the at least 10 discrete fluorescent signals from the 10 discrete locations on the substrate, each of the 10 discrete apertures being oriented to pass in-focus light from a different one of the at least 10 discrete fluorescent signals.

102. The system of claim 100, wherein the confocal filter comprises at least 1000 discrete confocal apertures positioned in a focal plane of an image of the at least 1000 discrete fluorescent signals from the 10 discrete locations on the substrate, each of the 1000 discrete apertures being oriented to pass in-focus light from a different one of the at least 1000 discrete fluorescent signals.

103. The system of claim 100, wherein the confocal filter comprises at least 5000 discrete confocal apertures positioned in a focal plane of an image of the at least 5000 discrete fluorescent signals from the 10 discrete locations on the substrate, each of the 5000 discrete apertures being oriented to pass in-focus light from a different one of the at least 5000 discrete fluorescent signals.

104. The system of claim 100, wherein a field lens is positioned between the first pair of tunable lenses to refocus confocally filtered fluorescence onto the detector.

105. The system of claim 95, wherein each member lens of the first pair of tunable lenses comprises at least two lenses.

106. The system of claim 105, wherein the at least two lenses comprise a doublet.

107. The system of claim 99, wherein the optical train further comprises a second pair of tunable lenses.

108. The system of claim 107, wherein the first pair of tunable lenses is provided in the optical path between the excitation illumination source and the substrate, and the second pair of tunable lenses is provided in the optical path between the substrate and the detector.

109. The system of claim 95, wherein the first DOE converts the single originating illumination beam from the first excitation illumination source into at least 10, at least 100, at least 500, at least 1000, or at least 5000 discrete illumination beams, each beam being directed at a different one of the fluorescent signal sources on the substrate.

110. The system of claim 95, wherein the plurality of discrete illumination beams each propagate at a unique angle relative to the single originating illumination beam from the first excitation illumination source.

111. The system of claim 95 wherein the plurality of discrete illumination beams have different power levels.

112. The system of claim 95, wherein the plurality of discrete illumination beams are oriented in a two-dimensional array of beams.

113. The system of claim 95, wherein the optical train further comprises a microlens array or a plurality of optical fibers to simultaneously direct excitation illumination at the plurality of discrete fluorescent signal sources on the substrate.

114. The system of claim 95, wherein each of the plurality of discrete fluorescent signal sources comprises a reaction region having disposed therein a complex of a nucleic acid polymerase, a template sequence, and a primer sequence, and at least one fluorescently labeled nucleotide.

115. The system of claim 95, wherein the reaction region comprises an optically confined region or a zero-mode waveguide on the substrate.

116. The system of claim 95, the system comprising:
at least a second excitation illumination sources that provides light at a second spectrum different from the first spectrum; and,
a second diffractive optical element (DOE) that converts a single originating illumination beam from the second excitation illumination source into a second plurality of discrete illumination beams, each beam from the second plurality being directed at a different one of the plurality of discrete fluorescent signal sources on the substrate.

117. A method of detecting a plurality of discrete fluorescent signals from a plurality of discrete fluorescent signal sources, the method comprising:
providing the system of claim 95;
simultaneously directing excitation illumination from the first excitation illumination source at the plurality of discrete fluorescent signal sources on the substrate in a targeted illumination pattern;
collecting the plurality discrete fluorescent signals simultaneously from the plurality of discrete signal sources with the optical train;
filtering the discrete fluorescent signals to reduce fluorescence not in the first focal plane to provide filtered fluorescent signals; and,
detecting the filtered fluorescent signals with the detector.

118. A method of reducing fluorescence background noise in monitoring fluorescent signals from at least one fluorescent signal source, the method comprising:
providing an excitation illumination source, a substrate having the at least one fluorescent signal source disposed thereon, and an optical train comprising optical components, which optical train is positioned to direct excitation illumination from the illumination source to the at least first fluorescent signal source and transmit fluorescent signals from the at least first fluorescent signal source to a detector;
photobleaching at least one of the optical components to reduce an amount of autofluorescence background noise produced by the at least one optical component in response to the excitation illumination;
directing excitation illumination through the at least one optical component and at the at least one fluorescent signal source; and,
detecting the fluorescent signals from the at least first fluorescent signal source.
